SEQUENCE LISTING (56 sequences)

SEQ ID NO: 1 - DNA, 1561 bp, Homo sapiens
taattccagctactctggaggctgaggcaggaggatggcttgagcccaggagttggaggctgcagttagccatgcttgtgccactacactccagcccgggcaacagggcaagactctgtatctaaaaacaacaacaacaacaataatagaaacaggtttcctttcccaagtttggaaaatctggtagtcttcttaagcagccatgagcataaagagaggattgttcataccacaggtgttccaggcataacgaaactgtctttgtgtttagttacaaggagaacatcatgcgcctctccagccttcacaaggaccgcccggtggagccgctggacctggccgtgttctgggtggagtttgtgatgaggcacaagggcgcgccacacctgcgccccgcagcccacgacctcacctggtaccagtaccattccttggacgtgattggtttcctcttggccgtcgtgctgacagtggccttcatcacctttaaatgttgtgcttatggctaccggaaatgcttggggaaaaaagggcgagttaagaaagcccacaaatccaagacccattgagaagtgggtgggaaataaggtaaaattttgaaccattccctagtcatttccaaacttgaaaacagaatcagtgttaaattcattttattcttattaaggaaatactttgcataaattaatcagccccagagtgctttaaaaaattctcttaaataaaaataatagactcgctagtcagtaaagatatttgaatatgtatcgtgccccctctggtgtctttgatcaggatgacatgtgccatttttcagaggacgtgcagacaggctggcattctagattacttttcttactctgaaacatggcctgtttgggagtgcgggattcaaaggtggtcccacggctgcccctactgcaaatggcagttttaatcttatcttttggcttctgcagatggttgcaattgatccttaaccaataatggtcagtcctcatctctgtcgtgcttcataggtgccaccttgtgtgtttaaagaagggaagctttgtacctttagagtgtaggtgaaatgaatgaatggcttggagtgcactgagaacagcatatgatttcttgctttggggaaaaagaatgatgctatgaaattggtgggtggtgtatttgagaagataatcattgcttatgtcaaatggagctgaatttgataaaaacccaaaatacagctatgaagtgctgggcaagtttactttttttctgatgtttcctacaactaaaaataaattaataaatttatataaattctatttaagtgttttcactggtgtcgcatttatttcttgttaagttgcattttctaattacaaaagtaatgcatgattatgacagaaagtttggaaaatatagaggttcacacacacacgccttcattgcgtgtgcatgcataaatgcatgagaaaagaaaaataaccagtaatcacatcgcccagaaataaccccagttacaattgtggcaaatacacatacttataaatattgcagatatattaagtatacc

SEQ ID NO: 2 - DNA, 1220 bp, Homo sapiens
tgacacggccatagttggttcatattaaccatgtgattaaaatggttaaatattaatttgggttcttacatatcaaagggtaaaattcagagcaagggagaggtagacaggacctgtgaaaagcagtggttagtttagggaaaatacctaggagccctgtgatttggagagtgaaaactctttattaccgttgttactttaactctttccaggatggcctgcctccttcgctcatttcagagaatttctgcaggggttttcttcttagcactttggggcatggttgtaggtgacaagctgctggtggtccctcaggacggaagccactggcttagtatgaaggatatagttgaggttctcagtgaccggggtcatgagattgtagtggtggtgcctgaagttaatttgcttttgaaagaatccaaatactacacaagaaaaatctatccagtgccgtatgaccaagaagagctgaagaaccgttaccaatcatttggaaacaatcactttgctgagcgatcattcctaactgctcctcagacagagtacaggaataacatgattgttattggcctgtacttcatcaactgccagagcctcctgcaggacagggacaccctgaacttctttaaggagagcaagtttgatgctcttttcacagacccagccttaccctgtggggtgatcctggctgagtatttgggcctaccatctgtgtacctcttcaggggttttccgtgttccctggagcatacattcagcagaagcccagaccctgtgtcctacattcccaggtgctacacaaagttttcagaccacatgactttttcccaacgagtggccaacttccttgttaatttgttggagccctatctattttattgtctgttttcaaagtatgaagaactcgcatcagctgtcctcaagagagatgtggatataatcaccttatatcagaaggtctctgtttggctgttaagatatgactttgtgcttgaatatcctaggccggtcatgcccaacatggtcttcattggaggtatcaactgtaagaagaggaaagacttgtctcaggttggtgggtttatttcttttggactgccttgtttcttccaggctctgtcctccctcactcatttggctccttgagccgactgtcccttggaggatttcctggagaacggtggggggaagtgatacccggctcggagcagcgggaa

SEQ ID NO: 3 - DNA, 1279 bp, Homo sapiens
tgtattattatgagtaaatcattggcagtgaatgtgaatttttttttaaatgaatgaataagtacacgccttcttttgagggcaggttctatctgtacttcttccacttactatattataggagcttagaatcccagctgctggctctgggctgaagttctctgatggctcgtgcagggtggactggcctccttcccctatatgtgtgtctactgctgacctgtgctttgccaaggtcagggaagctgctggtagtgcccatggatgggagccactggttcaccatgcagtcggtggtggagaaactcatcctcagggggcatgaggtggtcgtagtcatgccagaggtgagttggcaactgggaagatcactgaattgcacagtgaagacttactcaacctcatacactctggaggatcaggaccgggagttcatggtttttgccgatgctcgctggacggcaccattgcgaagtgcattttctctattaacaagttcatccaatggtatttttgacttatttttttcaaattgcaggagtttgtttaatgaccgaaaattagtagaatacttaaaggagagttgttttgatgcagtgtttctcgatccttttgatcgctgtggcttaattgttgccaaatatttctccctcccctctgtggtcttcgccaggggaatattttgccactatcttgaagaaggtgcacagtgccctgctcctctttcctatgtccccagacttctcttagggttctcagacgccatgactttcaaggagagagtatggaaccacatcatgcacttggaggaacatttattttgcccctattttttcaaaaatgtcttagaaatagcctctgaaattctccaaacccctgtcacggcatatgatctctacagccacacatcaatttggttgttgcgaactgactttgttttggagtatcccaaacccgtgatgcccaatatgatcttcattggtggtatcaactgtcatcagggaaagccagtgcctatggtaagttatctcccctttagcacattaagaataatctggctttggaaattaaaagatttcttacagaatcataatttatcatttacatttgtcccatttggaatttctttctggtttaaggaattcttttgtaccaattcacttaattgttgggtagcaaattgtataaagcagctcttgttgatatgtaagtgtatacaattgatataattgtagatcatatctaggctgcaatctaaatgctatttttggaaaaatac

SEQ ID NO: 4 - DNA, 1452 bp, Homo sapiens
ctctccctccaaggcgaagaccataatctactcttgtctgaaatcatacaagtaggtatctcagcaaatgatactcgtgtgttatcgttcttatgagtaaatcattggcagtgagtgtgattttttttttttttatgaaaggataaatacacgccctctattggggtcaggttttgtgcctgtacttcttccgcctactgtatcatagcagcttagaatcccagctgctggctcgggctgcagttctctcatcgtcgcgcagggtgatggctcgcgcagggtggaccagccccgttcctttatgtgtgtgtctactgctgacctgtggctttgccgaggcagggaagctgctggtagtgcccatggatgggagtcactggttcaccatgcagtcggtggtggagaaacttatcctcagggggcatgaggtggttgtagtcatgccagaggtgagttggcaactggaaagatcactgaattgcacagtgaagacttactcaacctcgtacactctggaagatcagaaccgggaattcatggttttcgcccatgctcaatggaaagcacaggcacaaagtatattttctctattaatgagttcatccagtggttttcttgacttatttttttcgcattgcaggagtttgtttaatgaccgaaaattagtagaatacttaaaggagagttcttttgatgcagtgtttctggatccttttgatacctgtggcttaattgttgctaaatatttctccctcccctctgtggtcttcaccaggggaatattttgccaccatcttgaagaaggtgcacagtgccctgctcctctttcctatgtccccaatgatctcttagggttctcagatgccatgactttcaaggagagagtatggaaccacatcgtgcacttggaggaccatttattttgccagtatctttttagaaatgccctagaaatagcctctgaaattctccaaacccctgtcacggcatatgatctctacagtcacacatcaatttggttgttgcgaacggactttgttttggactatcccaaacccgtgatgcccaacatgatcttcattggtggtatcaactgtcatcagggaaagccattgcctatggtaagtcacctctcctttagcacattaagaataatctggctttggaattaaaaaaggattccttactgaactgtgatttgacatttcgttgtggcattcaatttctttccagtttaacaaattattttgtgcgaattcatgtactcatcaattatcaaattttataaaactgcccttcttgaaagtatatgtaataatttaaaaattatagatcatattcaggctacattttaaaatacgatgtttagaaaagtaccaaaaaaccacagcaagaaatgaaacttccgtttttttgttattctatgtgaccccgtagttgaaaatgctctta

SEQ ID NO: 5 - DNA, 1244 bp, Homo sapiens
gggcatgatctgtccaaggcagagactataagctactcttatagtactcttatgagatacatacaagtaggtatctcaaaaaatgatactcatgtattcctgttcttatgagtaaatcattggcagtgagtgtgattttttttttttttatgacaggatccctacacgccctctattggggtcaggttttgtgcctgtagttcttccgcctacgtatcatagcagttagaatcccagctgctggctcgggctgcagttctctcatggctcgcacagggtggaccagccccattcccctatgtgtttctctgctgctgacctgtggctttgctgaggcagggaagctgctggtagtgcccatggatgggagtcactggttcaccatgcagtcggtggtggagaaacttatcctcagggggcatgaggtggttgtagtcatgccagaggtgagttggcaactgggaaaatcactgaattgcacagtgaagacttactcaacctcatacactctggaggatctggaccgggaattcatggatttcgccgatgctcaatggaaagcacaagtacgaagtttgttttctctatttctgagttcatccaatggtttttttaacttatttttttcgcattgcaggagtttgtttaatgaccgaaaattagtagaatacttaaaggagagttcttttgatgcggtgtttcttgatccttttgatgcctgtgcgttaattgttgccaaatatttctccctcccctctgtggtcttcgccaggggaataggttgccactatcttgaagaaggtgcacagtgccctgctcctctttcctatgtccccagaattctcttagggttctcagatgccatgactttcaaggagagagtacggaaccacatcatgcacttggaggaacatttattttgccagtatttttccaaaaatgccctagaaatagcctctgaaattctccaaacacctgtcacagcatatgatctctacagccacacatcaatttggttgttgcgaacagactttgttttggactatcccaaacccgtgatgcccaatatgatcttcattggtggtatcaactgccatcagggaaagccattgcctatggtaagtcacctctcctttagcacattaggaataatcttggctttggaaattaaaaaaagattccttactgaattgtgatttgacattttcatttgttgcatttcaaatttctttccagtttacaga

SEQ ID NO: 6 - DNA, 2320 bp, Homo sapiens
ctcagctgcagttctctgatggcttgcacagggtggaccagcccccttcctctatgtgtgtgtctgctgctgacctgtggctttgccgaggcagggaagctactggtagtgcccatggatgggagccactggttcaccatgaggtcggtggtggagaaactcattctcagggggcatgaggtggttgtagtcatgccagaggtgagttggcaactgggaagatcactgaattgcacagtgaagacttattcaacttcatataccctggaggatctggaccgggagttcaaggcttttgcccatgctcaatggaaagcacaagtacgaagtatatattctctattaatgggttcatacaatgacatttttgacttatttttttcaaattgcaggagtttgtttaaagacaaaaaattagtagaatacttaaaggagagttcttttgatgcagtgtttctcgatccttttgataactgtggcttaattgttgccaaatatttctccctcccctccgtggtcttcgccaggggaatactttgccactatcttgaagaaggtgcacagtgccctgctcctctttcctatgtccccagaattctcttagggttctcagatgccatgactttcaaggagagagtacggaaccacatcatgcacttggaggaacatttattatgccaccgttttttcaaaaatgccctagaaatagcctctgaaattctccaaacacctgttacggagtatgatctctacagccacacatcaatttggttgttgcgaacggactttgttttggactatcccaaacccgtgatgcccaacatgatcttcattggtggtatcaactgccatcagggaaagccgttgcctatggaatttgaagcctacattaatgcttctggagaacatggaattgtggttttctctttgggatcaatggtctcagaaattccagagaagaaagctatggcaattgctgatgctttgggcaaaatccctcagacagtcctgtggcggtacactggaacccgaccatcgaatcttgcgaacaacacgatacttgttaagtggctaccccaaaacgatctgcttggtcacccgatgacccgtgcctttatcacccatgctggttcccatggtgtttatgaaagcatatgcaatggcgttcccatggtgatgatgcccttgtttggtgatcagatggacaatgcaaagcgcatggagactaagggagctggagtgaccctgaatgttctggaaatgacttctgaagatttagaaaatgctctaaaagcagtcatcaatgacaaaagttacaaggagaacatcatgcgcctctccagccttcacaaggaccgcccggtggagccgctggacctggccgtgttctgggtggagtttgtgatgaggcacaagggcgcgccacacctgcgccccgcagcccacgacctcacctggtaccagtaccattccttggacgtgattggtttcctcttggccgtcgtgctgacagtggccttcatcacctttaaatgttgtgcttatggctaccggaaatgcttggggaaaaaagggcgagttaagaaagcccacaaatccaagacccattgagaagtgggtgggaaataaggtaaaattttgaaccattccctagtcatttccaaacttgaaaacagaatcagtgttaaattcattttattcttattaaggaaatactttgcataaattaatcagccccagagtgctttaaaaaattctcttaaataaaaataatagactcgctagtcagtaaagatatttgaatatgtatcgtgccccctccggtgtctttgatcaggatgacatgtgccatttttcagaggacgtgcagacaggctggcattctagattacttttcttactctgaaacatggcctgtttgggagtgcgggattcaaaggtggtcccaccgctgcccctactgcaaatggcagttttaatcttatcttttggcttctgcagatggttgcaattgatccttaaccaataatggtcagtcctcatctctgtcctgcttcataggtgccaccttgtgtgtttaaagaagggaagctttgtacctttagagtgtaggtgaaatgaatgaatggcttggagtgcactgagaacagcatatgatttcttgctttggggaaaaagaatgatgctatgaaattggtgggtggtgtatttgagaagataatcattgcttatgtcaaatggagctgaatttgataaaaacccaaaatacagctatgaagtgctgggcaagtttactttttttctgatgtttcctacaact

SEQ ID NO: 7 - DNA, 512 bp, Homo sapiens
tttgcatctcaaggataattctgtaagcaggaacccttcctcctttagaaggaagtaaaggagaggaaaatgctgtaaaacttacatattaataattttttactctatctcaaacacgcatgcctttaatcatagtcttaagaggaagatatctaattcataacttactgtatgtagtcatcaaagaatatgagaaaaaattaactgaaaatttttcttctggctctaggaatttgaagcctacattaatgcttctggagaacatggaattgtggttttctctttgggatcaatggtctcagaaattccagagaagaaagctatggcaattgctgatgctttgggcaaaatccctcagacagtaagaagattctataccatggcctcatatctattttcacaggagcgctaatcccagacttccagcttccagattaattctcttaattggaaccttagatttggcttttccctgccacttcccaactattaatccaaaggttttttttgtt

SEQ ID NO: 8 - DNA, 784 bp, Homo sapiens
aaagatgtcctcaagggaccctgttttctagttagtatagcagatttgttttctaatcatattatgtctttctttacgttctgctcttttgcccctcccaggtcctgtggcggtacactggaacccgaccatcgaatcttgcgaacaacacgatacttgttaagtggctaccccaaaacgatctgcttggtatgttgggcggattggatgtataggtcaaaccagggtcaaattaagaaaatggcttaagcacagctattctaaaggattgttgagcttgaaaatattatggccaacatatcctacattgctttttatctagtggggtatctcaacccacattttcttctgcaaatttctgcaagggcatgtgagtaacactgagtctttggagtgttttcagaacctagatgtgtccagctgtgaaactcagagatgtaactgctgacatcctccctattttgcatctcaggtcacccgatgacccgtgcctttatcacccatgctggttcccatggtgtttatgaaagcatatgcaatggcgttcccatggtgatgatgcccttgtttggtgatcagatggacaatgcaaagcgcatggagactaagggagctggagtgaccctgaatgttctggaaatgacttctgaagatttagaaaatgctctaaaagcagtcatcaatgacaaaaggtaagaaagaagatacagaagaatactttggtcatggcattcatgataaaattgtttcaaatatgaaaacatttacgtagcatttaatacgt

SEQ ID NO: 9 - DNA, 18 bp, Homo sapiens
gcacaccttgtccttcat

SEQ ID NO: 10 - DNA, 18 bp, Homo sapiens
gcacaccttgtccttcac

SEQ ID NO: 11 - DNA, 19 bp, Homo sapiens
catcaccatcgagatcaac

SEQ ID NO: 12 - DNA, 20 bp, Homo sapiens
ctattaaatcgcctctctct

SEQ ID NO: 13 - DNA, 20 bp, Homo sapiens
ctattaaatcgcctctctcc

SEQ ID NO: 14 - DNA, 19 bp, Homo sapiens
ggatgaatttcaagtattt

SEQ ID NO: 15 - DNA, 18 bp, Homo sapiens
agcccagataccccaaag

SEQ ID NO: 16 - DNA, 18 bp, Homo sapiens
agcccagataccccaaaa

SEQ ID NO: 17 - DNA, 18 bp, Homo sapiens
tgtgctttctcgtcttca

SEQ ID NO: 18 - DNA, 18 bp, Homo sapiens
tgtaattcctgccgtcag

SEQ ID NO: 19 - DNA, 18 bp, Homo sapiens
tgtaattcctgccgtcaa

SEQ ID NO: 20 - DNA, 17 bp, Homo sapiens
atacagcactggcatgg

SEQ ID NO: 21 - DNA, 18 bp, Homo sapiens
cctggagtttgtggggcg

SEQ ID NO: 22 - DNA, 18 bp, Homo sapiens
cctggagtttgtggggca

SEQ ID NO: 23 - DNA, 18 bp, Homo sapiens
tgaaccatgaagtccacg

SEQ ID NO: 24 - DNA, 18 bp, Homo sapiens
cgtgttccctggagcata

SEQ ID NO: 25 - DNA, 18 bp, Homo sapiens
cgtgttccctggagcatg

SEQ ID NO: 26 - DNA, 20 bp, Homo sapiens
gaatgtaggacacagggtct

SEQ ID NO: 27 - DNA, 18 bp, Homo sapiens
gacacagggtctgggctt

SEQ ID NO: 28 - DNA, 18 bp, Homo sapiens
gacacagggtctgggctg

SEQ ID NO: 29 - DNA, 19 bp, Homo sapiens
tacctcttcaggggttttc

SEQ ID NO: 30 - DNA, 16 bp, Homo sapiens
tgcagtaggggcagcg

SEQ ID NO: 31 - DNA, 16 bp, Homo sapiens
tgcagtaggggcagcc

SEQ ID NO: 32 - DNA, 16 bp, Homo sapiens
ggagtgcgggattcaa

SEQ ID NO: 33 - DNA, 20 bp, Homo sapiens
acacggccatagttggttca

SEQ ID NO: 34 - DNA, 20 bp, Homo sapiens
cagttgatgaagtacaggcc

SEQ ID NO: 35 - DNA, 20 bp, Homo sapiens
tgtagtggtggtgcctgaag

SEQ ID NO: 36 - DNA, 21 bp, Homo sapiens
gacagctgatgcgagttcttc

SEQ ID NO: 37 - DNA, 20 bp, Homo sapiens
gagggcaggttctatcgtac

SEQ ID NO: 38 - DNA, 20 bp, Homo sapiens
gggcactgtgcaccttcttc

SEQ ID NO: 39 - DNA, 20 bp, Homo sapiens
acggcaccattgcgaagtgc

SEQ ID NO: 40 - DNA, 23 bp, Homo sapiens
acttacatatcaacaagtgctgc

SEQ ID NO: 41 - DNA, 20 bp, Homo sapiens
gggcatgatctgtccaaggc

SEQ ID NO: 42 - DNA, 22 bp, Homo sapiens
ggttgagtaagtcttcactgtg

SEQ ID NO: 43 - DNA, 18 bp, Homo sapiens
ctcagctgcagttctctg

SEQ ID NO: 44 - DNA, 21 bp, Homo sapiens
ccagatcctccagggtatatg

SEQ ID NO: 45 - DNA, 20 bp, Homo sapiens
gagttcatccagtggttttc

SEQ ID NO: 46 - DNA, 18 bp, Homo sapiens
cagttcagtaaggaatcc

SEQ ID NO: 47 - DNA, 22 bp, Homo sapiens
caaggataattctgtaagcagg

SEQ ID NO: 48 - DNA, 22 bp, Homo sapiens
ggattaatagttgggaagtggc

SEQ ID NO: 49 - DNA, 20 bp, Homo sapiens
ggccaacatatcctacattg

SEQ ID NO: 50 - DNA, 23 bp, Homo sapiens
cgtattaaatgctacgtaaatgt

SEQ ID NO: 51 - DNA, 20 bp, Homo sapiens
cagttagccatgcttgtgcc

SEQ ID NO: 52 - DNA, 20 bp, Homo sapiens
gcactctggggctgattaat

SEQ ID NO: 53 - DNA, 19 bp, Homo sapiens
cgtgctgacagtggccttc

SEQ ID NO: 54 - DNA, 20 bp, Homo sapiens
cagtgcactccaagccattc

SEQ ID NO: 55 - DNA, 18 bp, Homo sapiens
gatggttgcaattgatcc

SEQ ID NO: 56 - DNA, 20 bp, Homo sapiens
ttagttgtaggaaacatcag

(19) United States
(12) Patent Application Publication
(10) Pub. No.: US 2004/0076968 A1
(43) Pub. Date: Apr. 22, 2004

(54) Method for detecting pre-disposition to hepatotoxicity

(76) Inventors: Gonzalo Acuna, Dietikon (CH); Dorothee Foernzler, Lenzburg (CH); Diane Uratsu Leong, Berkeley, CA (US)

Correspondence Address: HELLER EHRMAN WHITE & MCAULIFFE LLP, 275 MIDDLEFIELD ROAD, MENLO PARK, CA, 94025-3506, US

(21) Appl. No.: 10/333,108

(22) PCT Filed: Jul. 2, 2001
(86) PCT No.: PCT/EP01/07524

(30) Foreign Application Priority Data
Jul. 14, 2000 (EP) 00115353.5

(51) Int. Cl. C12Q 1/68

(52) U.S. Cl. 435/6

(57) ABSTRACT

This invention relates to a method for diagnosing a pre-disposition to drug induced livertoxicity which method comprises determining at least one single nucleotide polymorphism in the UDP-glucuronosyl transferase (UGT1) gene. Said method is based on determining specific single nucleotide polymorphisms in the UGT1 gene in a human being and determining the status of said human being by reference to polymorphism in UGT1. The invention further relates to diagnostic nucleic acids comprising within their sequence the polymorphisms as defined herein, to allele-specific primers and allele-specific oligonucleotide probes capable of hybridizing to such diagnostic nucleic acids and to diagnostic kits comprising one or more of such primers and probes for detecting a poylymorphism in the UGT1 gene.

The present invention relates to a method for diagnosing a pre-disposition to drug induced livertoxicity which method comprises determining the polymorphisms in the UDP-glucuronosyl transferase (UGT1) gene.

UGT1 is a member of the UDP glucuronosyltransferase (UGT) gene superfamily. UGT enzymes catalyze the addition of the glucuronosyl group from a nucleotide sugar to a small hydrophobic molecule (aglycone) in order to enhance the water solubility of endo- and xenobiotics. UGT enzymes are involved in the metabolism of a large number of drugs. For a review on this enzyme superfamily see Pharmacogenetics (1997) 7, 255-269. The presence of at least nine UDP-glucuronosyl transferase isoenzymes has been described in International patent application WO 92/12987.

One of these enzymes, the human bilirubin glucuronosyl transferase gene encoded at the UGT1 locus has been associated with gene defects by Ritter et al., J. Clin. Invest. (1992), 90, 150-155; Aomoo et al., Biochem. Biophys. Res. Commun. (1993), 197, 1239-1244; Moghrabi et al., Am. J. Hum. Genet. (1993), 53, 722-729; Labrune et al., Hum. Genet. (1994), 94, 693-697 and Seppen et al. J. Clin. Invest. (1994), 268, 2385-2391.

The term polymorphism relates to the observation that different nucleotides can occur at a given position in a specific DNA sequence. Genetic polymorphisms occur at random throughout the genome. Genetic polymorphisms may affect the function of a gene by altering the structure of the protein that the gene codes or by affecting the level of expression of that gene.

Genetic variations or polymorphisms among individuals are responsible to a great extent for the observable biological differences between individuals. Genetic variations are also responsible for the differences on how individuals respond to a drug.

In vitro experiments have shown that variations in UGT1A6 and UGT1A7 genes affect the enzymatic activity on specific substrates of the enzymes coded by these genes.

Genetic polymorphisms in the human UGT1A6 (plasma phenol) UDP-glucuronosyl transferase and the pharmacological implications thereof have been described by Ciotti et al., Pharmacogenetics (1997),7, 485-495. The cloned and isolated UGT1A6*2 allelic variant (which contains the Thr181Ala and Arg 184Ser mutations) when expressed in COS cells metabolised, at pH 6.4, the substrates 4-nitrophenol, 4-tert-butylphenol, 3-ethylphenol, 4-ethylphenol, 4-hydroxycoumarin, butylated hydroxy anisole and butylated hydroxy toluene at only 27-75% of the rate of the wild-type isoenzyme. 1-Naphtol, 3iodophenol, 7-hydroxycoumarin, and 7-hydroxy-4-methylcoumarin were metabolised at normal levels. 3-O-Methyl-dopa and methyl salicylate were metabolised at 41-74% and β-blockers at 28-69% of the rate of the wild-type isoenzyme.

Guillemete et al., Pharmacogenetics (2000), 10, 629-644, showed that three different cloned and isolated allelic variants of UGT1A7, when expressed in HEK cells, showed different catalytic activity towards the substrates 3-, 7-and 9-hydroxy-benzo-(a)-pyrene as compared to the wild-type enzyme. UGT1A7*3 (Lys 129 Lys131 Arg208) exhibited 5.8-fold lower Vₘₐₓ relative to the wild-type UGT1A7*1 (Asn129 Arg131 Trp208), whereas UGT1A7*2 (Lys129 Lys131 Trp208) and UGT1A7*4 (Asn129 Arg131 Arg208) had a 2.6 and 2.8-fold lower relative Vₘₐₓ than UGT1A7*1. While the results mentioned above indicate that genetic variations in the UGT1 genes, when cloned and expressed in cultured cells, can affect the enzymatic activity of the corresponding gene products on a particular substrate, they show no proof that these variations have a significant effect on the metabolism of these substrates in human individuals.

It has now been found that specific genetic variations within the UGT1 gene complex, including UGT1A6 and UGT1A7, affect the response of individual human patients to a drug which is metabolised by these enzymes.

Pharmacogenetics is an approach to use the knowledge of polymorphisms to study the role of genetic variation among individuals in variation to drug response, a variation that often results from individual differences in drug metabolism. Pharmacogenetics helps to identify patients most suited to therapy with particular pharmaceutical agents. This approach can be used in pharmaceutical research to assist the drug selection process. Polymorphisms are used in mapping the human genome and to elucidate the genetic component of diseases. Details on pharmacogenetics and other uses of polymorphism detection can be found in Linder et al. (1997), Clinical Chemistry, 43, 254; Marshall (1997), Nature Biotechnology, 15, 1249; International Patent Application WO 97/40462, Spectra Biomedical; and Schafer et al. (1998), Nature Biotechnology, 16, 33.

As particular mutations or polymorphisms associated with certain clinical features, such as adverse or abnormal events, are likely to be of low frequency within the population, low frequency SNPs may be particularly usefll in identifying these mutations (for examples see: Linkage disequilibrium at the cystathionine beta synthase (CBS) locus and the association between genetic variation at the CBS locus and plasma levels of homocysteine (De Stefano et al., Ann. Hum. Genet. (1998) 62, 481-90). Variation at the von Willebrand factor (vWF) gene locus is associated with plasma vWF:Ag levels: identification of three novel single nucleotide polymorphisms in the vWF gene promoter (Keightley et al., Blood (1999) 93, 4277-83).

It has been found that in rare cases the administration of pharmaceutically active agents to human beings leads to hepatotoxicity. A typical example is the occurrence of reversible asymptomatic increase in liver transaminases activity found in certain patients with Parkinson's disease (PD) who had participated in clinical trials for tolcapone. The studies indicated that in rare circumstances, tolcapone could induce a reversible asymptomatic increase in liver transaminase activity. There was therefore a desire to establish whether there is a correlation between the occurrence of such liver abnormalities, which are indicators of liver toxicity and certain genetic pre-dispositions. It has now been found that variations in genes involved in tolcapone (TASMAR) metabolism and pharmacology cause abnormalities in metabolic activity in certain individuals, resulting in an accumulation in the liver of this drug or its metabolites to toxic levels.

Clinical trials have shown that patient response to treatment with pharmaceuticals is often heterogeneous. Thus there is also a need for improved approaches to pharmaceutical agent design and therapy.

The present invention therefore provides a genetic diagnostic tool for identifying the pre-disposing genotypes. Said tool consists of a method for detecting a predisposition to a hepatotoxic reaction caused by the administration of a pharmaceutically active compound to a human being based on the determination of at least one single nudeotide polymorphism in the UDP-glucuronosyltransferase (UGT1) gene in the sample of said human being, which method comprises determining the nucleotide at position 908 in exon 5 of the UGT1 gene as defined by the position in SEQ ID NO: 1 and determining the status of the human being by reference to polymorphism in UGT1. Alternatively or, in addition thereto, the method comprises determining the sequence of the nucleic acid of the human being at position 528 in exon 1 of the UGT1A6 gene as defined by SEQ ID NO: 2 or determing the sequence of the nucleic acid of the human being at position 197 in exon 1 of the UGT1A7 gene as defined by sequence ID NO: 3 and determining the status of said human being by reference to polymorphism in UGT1.

An individual possesses a predisposition to a hepatotoxic reaction when his UGT1 gene contains variations which lead to abnormalities in its metabolic activity.

SEQ ID NO: 1 refers to Genbank accession number M84124, which provides exon 5 of UGT1:

| | |
|---|---|
| 1 | TAATTCCAGC TACTCTGGAG GCTGAGGCAG GAGGATGGCT TGAGCCCAGG |
| 51 | AGTTGGAGGC TGCAGTTAGC CATGCTTGTG CCACTACACT CCAGCCCGGG |
| 101 | CAACAGGGCA AGACTCTGTA TCTAAAAACA ACAACAACAA CAATAATAGA |
| 151 | AACAGGTTTC CTTTCCCAAG TTTGGAAAAT CTGGTAGTCT TCTTAAGCAG |
| 201 | CCATGAGCAT AAAGAGAGGA TTGTTCATAC CACAGGTGTT CCAGGCATAA |
| 251 | CGAAACTGTC TTTGTGTTTA GTTACAAGGA GAACATCATG CGCCTCTCCA |
| 301 | GCCTTCACAA GGACCGCCCG GTGGAGCCGC TGGACCTGGC CGTGTTCTGG |
| 351 | GTGGAGTTTG TGATGAGGCA CAAGGGCGCG CCACACCTGC GCCCCGCAGC |
| 401 | CCACGACCTC ACCTGGTACC AGTACCATTC CTTGGACGTG ATTGGTTTCC |
| 451 | TCTTGGCCGT CGTGCTGACA GTGGCCTTCA TCACCTTTAA ATGTTGTGCT |
| 501 | TATGGCTACC GGAAATGCTT GGGGAAAAAA GGGCGAGTTA AGAAAGCCCA |
| 551 | CAAATCCAAG ACCCATTGAG AAGTGGGTGG GAAATAAGGT AAAATTTTGA |
| 601 | ACCATTCCCT AGTCATTTCC AAACTTGAAA ACAGAATCAG TGTTAAATTC |
| 651 | ATTTTATTCT TATTAAGGAA ATACTTTGCA TAAATTAATC AGCCCCAGAG |
| 701 | TGCTTTAAAA AATTCTCTTA AATAAAAATA ATAGACTCGC TAGTCAGTAA |
| 751 | AGATATTTGA ATATGTATCG TGCCCGCTCT GGTGTCTTTG ATCAGGATGA |
| 801 | CATGTGCCAT TTTTCAGAGG ACGTGCAGAC AGGCTGGCAT TCTAGATTAC |
| 851 | TTTTCTTACT CTGAAACATG GCCTGTTTGG GAGTGCGGGA TTCAAAGGTG |
| 901 | GTCCCACGGC TGCCCCTACT GCAAATGGCA GTTTTAATCT TATCTTTTGG |
| 951 | CTTCTGCAGA TGGTTGCAAT TGATCCTTAA CCAATAATGG TCAGTCCTCA |
| 1001 | TCTCTGTCGT GCTTCATAGG TGCCACCTTG TGTGTTTAAA GAAGGGAAGC |
| 1051 | TTTGTACCTT TAGAGTGTAG GTGAAATGAA TGAATGGCTT GGAGTGCACT |
| 1101 | GAGAACAGCA TATGATTTCT TGCTTTGGGG AAAAAGAATG ATGCTATGAA |
| 1151 | ATTGGTGGGT GGTGTATTTG AGAAGATAAT CATTGCTTAT GTCAAATGGA |
| 1201 | GCTGAATTTG ATAAAAACCC AAAATACAGC TATGAAGTGC TGGGCAAGTT |
| 1251 | TACTTTTTTT CTGATGTTTC CTACAACTAA AAATAAATTA ATAAATTTAT |
| 1301 | ATAAATTCTA TTTAAGTGTT TTCACTGGTG TCGCATTTAT TTCTTGTTAA |
| 1351 | GTTGCATTTT CTAATTACAA AAGTAATGCA TGATTATGAC AGAAAGTTTG |
| 1401 | GAAAATATAG AGGTTCACAC ACACACGCCT TCATTGCGTG TGCATGCATA |
| 1451 | AATGCATGAG AAAAGAAAAA TAACCAGTAA TCACATCGCC CAGAAATAAC |
| 1501 | CCCAGTTACA ATTGTGGCAA ATACACATAC TTATAAATAT TGCAGATATA |
| 1551 | TTAAGTATAC C |

The present invention is based on the discovery of novel single nucleotide polymorphisms (SNP) in the UGT1 gene locus, viz.

at position 908 of SEQ ID NO: 1. The polymorphism at this position consists of the replacement of the nucleotide G at this position by a C in exon 5 of the UGT1 gene locus;

at position 528 of SEQ ID NO: 2. The polymorphism at this position consists of the replacement of the nucleotide A at this position by a G in exon 1 of the UGT1A6 gene;

and at position 197 of SEQ ID NO: 3. The polymorphism at this position consists of the replacement of the nucleotide C at this position by a G in exon 1 of the UGT1A7 gene.

As defined herein, the UGT1 gene includes exon coding sequences for all different UGT1A isozymes, intron sequences intervening the exon sequences and 3′ and 5′ untranslated region (3′ UTR and 5′ UTR) sequences, including the promoter element of the UGT1 gene, encoding for all UGT1A isozymes.

SEQ ID NO: 2 refers to Genbank accession number M84130, which provides exon 1 of UGT1A6:

| | |
|---|---|
| 1 | TGACACGGCC ATAGTTGGTT CATATTAACC ATGTGATTAA AATGGTTAAA |
| 51 | TATTAATTTG GGTTCTTACA TATCAAAGGG TAAAATTCAG AGCAAGGGAG |
| 102 | AGGTAGACAG GACCTGTGAA AAGCAGTGGT TAGTTTAGGG AAAATACCTA |
| 151 | GGAGCCCTGT GATTTGGAGA GTGAAAACTC TTTATTACCG TTGTTACTTT |
| 201 | AACTCTTTCC AGGATGGCCT GCCTCCTTCG CTCATTTCAG AGAATTTCTG |
| 251 | CAGGGGTTTT CTTCTTAGCA CTTTGGGGCA TGGTTGTAGG TGACAAGCTG |
| 301 | CTGGTGGTCC CTCAGGACGG AAGCCACTGG CTTAGTATGA AGGATATAGT |
| 351 | TGAGGTTCTC AGTGACCGGG GTCATGAGAT TGTAGTGGTG GTGCCTGAAG |
| 401 | TTAATTTGCT TTTGAAAGAA TCCAAATACT ACACAAGAAA AATCTATCCA |
| 451 | GTGCCGTATG ACCAAGAAGA GCTGAAGAAC CGTTACCAAT CATTTGGAAA |
| 501 | CAATCACTTT GCTGAGCGAT CATTCCTAAC TGCTCCTCAG ACAGACTACA |
| 551 | GGAATAACAT GATTGTTATT GGCCTGTACT TCATCAACTG CCAGAGCCTC |
| 601 | CTGCAGGACA GGGACACCCT GAACTTCTTT AAGGAGAGCA AGTTTGATGC |
| 651 | TCTTTTCACA GACCCAGCCT TACCCTGTGG GGTGATCCTG GCTGAGTATT |
| 701 | TGGGCCTACC ATCTGTGTAC CTCTTCAGGG GTTTTCCGTG TTCCCTGGAG |
| 751 | CATACATTCA GCAGAAGCCC AGACCCTGTG TCCTACATTC CCAGGTGCTA |
| 801 | CACAAAGTTT TCAGACCACA TGACTTTTTC CCAACGAGTG GCCAACTTCC |
| 851 | TTGTTAATTT GTTGGAGCCC TATCTATTTT ATTGTCTGTT TTCAAAGTAT |
| 901 | GAAGAACTCG CATCAGCTGT CCTCAAGAGA GATGTGGATA TAATCACCTT |
| 951 | ATATCAGAAG GTCTCTGTTT GGCTGTTAAG ATATGACTTT GTGCTTGAAT |
| 1001 | ATCCTAGGCC GGTCATGCCC AACATGGTCT TCATTGGAGG TATCAACTGT |
| 1051 | AAGAAGAGGA AAGACTTGTC TCAGGTTGGT GGGTTTATTT CTTTTGGACT |
| 1101 | GCCTTGTTTC TTCCAGGCTC TGTCCTCCCT CACTCATTTG GCTCCTTGAG |
| 1151 | CCGACTGTCC CTTGGAGGAT TTCCTGGAGA ACGGTGGGGG GAAGTGATAC |
| 1201 | CCGGCTCGGA GCAGCGGGAA |

SEQ ID NO: 3 refers to Genbank accession number U39570, which provides exon 1 of UGT1A7:

| | |
|---|---|
| 1 | TGTATTATTA TGAGTAAATC ATTGGCAGTG AATGTGAATT TTTTTTTAAA |
| 51 | TGAATGAATA AGTACACGCC TTCTTTTGAG GGCAGGTTCT ATCTGTACTT |
| 101 | CTTCCACTTA CTATATTATA GGAGCTTAGA ATCCCAGCTG CTGGCTCTGG |
| 151 | GCTGAAGTTC TCTGATGGCT CGTGCAGGGT GGACTGGCCT CCTTCCCCTA |
| 201 | TATGTGTGTC TACTGCTGAC CTGTGCTTTG CCAAGGTCAG GGAAGCTGCT |
| 251 | GGTAGTGCCC ATGGATGGGA GCCACTGGTT CACCATGCAG TCGGTGGTGG |
| 301 | AGAAACTCAT CCTCAGGGGG CATGAGGTGG TCGTAGTCAT GCCAGAGGTG |
| 351 | AGTTGGCAAC TGGGAAGATC ACTGAATTGC ACAGTGAAGA CTTACTCAAC |
| 401 | CTCATACACT CTGGAGGATC AGGACCGGGA GTTCATGGTT TTTGCCGATG |
| 451 | CTCGCTGGAC GGCACCATTG CGAAGTGCAT TTTCTCTATT AACAAGTTCA |
| 501 | TCCAATGGTA TTTTTGACTT ATTTTTTTCA AATTGCAGGA GTTTGTTTAA |
| 551 | TGACCGAAAA TTAGTAGAAT ACTTAAAGGA GAGTTGTTTT GATGCAGTGT |
| 601 | TTCTCGATCC TTTTGATCGC TGTGGCTTAA TTGTTGCCAA ATATTTCTCC |
| 651 | CTCCCCTCTG TGGTCTTCGC CAGGGGAATA TTTTGCCACT ATCTTGAAGA |
| 701 | AGGTGCACAG TGCCCTGCTC CTCTTTCCTA TGTCCCCAGA CTTCTCTTAG |
| 751 | GGTTCTCAGA CGCCATGACT TTCAAGGAGA GAGTATGGAA CCACATCATG |
| 801 | CACTTGGAGG AACATTTATT TTGCCCCTAT TTTTTCAAAA ATGTCTTAGA |
| 851 | AATAGCCTCT GAAATTCTCC AAACCCCTGT CACGGCATAT GATCTCTACA |
| 901 | GCCACACATC AATTTGGTTG TTGCGAACTG ACTTTGTTTT GGAGTATCCC |
| 951 | AAACCCGTGA TGCCCAATAT GATCTTCATT GGTGGTATCA ACTGTCATCA |
| 1001 | GGGAAAGCCA GTGCCTATGG TAAGTTATCT CCCCTTTAGC ACATTAAGAA |
| 1051 | TAATCTGGCT TTGGAAATTA AAAGATTTCT TACAGAATCA TAATTTATCA |
| 1101 | TTTACATTTG TCCCATTTGG AATTTCTTTC TGGTTTAAGG AATTCTTTTG |
| 1151 | TACCAATTCA CTTAATTGTT GGGTAGCAAA TTGTATAAAG CAGCTCTTGT |
| 1201 | TGATATGTAA GTGTATACAA TTGATATAAT TGTAGATCAT ATCTAGGCTG |
| 1251 | CAATCTAAAT GCTATTTTTG GAAAAATAC |

Furthermore the invention relates to a method for detecting a predisposition to liver toxicity after administration of a pharmaceutically active compound based on one or more single nucleotide polymorphism(s) in the UDP-glucuronosyltransferase (UGT1) gene locus in a human being, wherein additionally the polymorphism at one of the following positions is determined:

232 in exon 1 of UGT1A6 as defined by the position of SEQ ID NO: 2;

754 in exon 1 of UGT1A6 as defined by the position in SEQ ID NO: 2,

765 in exon 1 of UGT1A6 as defined by the position in SEQ ID NO: 2;

551 in exon 1 of UGT1A7 as defined by the position in SEQ ID NO: 3;

555 in exon 1 of UGT1A7 as defined by the position in SEQ ID NO: 3;

556 in exon 1 of UGT1A7 as defined by the position in SEQ ID NO: 3; or

786 in exon 1 of UGT1A7 as defined by the position in SEQ ID NO: 3.

The polymorphism at position 232 in exon 1 of UGT1A6 of SEQ ID NO: 2 consists of a replacement of the nucleotide T at this position by a G (which results in a Ser to Ala amino acid exchange at position 7 in the corresponding protein).

The polymorphism at position 754 of SEQ ID NO: 2 consists of a replacement of the nucleotide A at this position by a G in exon 1 of UGT1A6 (which results in a Thr to Ala amino acid exchange at position 181 in the corresponding protein).

The polymorphism at position 765 of SEQ ID NO: 2 consists of a replacement of the nucleotide A at this position by a C in exon 1 of UGT1A6 (which corresponds to a Arg to Ser amino acid exchange at position 184 of the corresponding protein).

The polymorphism at position 551 of SEQ ID NO: 3 consists of the replacement of the nucleotide T at this position by a G in exon 1 of the UGT1A7 gene and results in an amino acid exchange from Asn to Lys at position 129 in the corresponding protein.

The polymorphism at position 555 of SEQ ID NO: 3 consists of the replacement of the nucleotide C at this position by an A in exon 1 of the UGT1A7 gene. This may result in a silent mutation (if the nucleotide at position 556 of SEQ ID NO: 3 consists of a G) or result in an amino acid exchange from Arg to Lys at position 131 in the corresponding protein (if the nucleotide at position 556 of SEQ ID NO: 3 consists of an A).

The polymorphism at position 556 of SEQ ID NO: 3 consists of the replacement of the nucleotide G at this position by an A in exon 1 of the UGT1A7 gene and results in an amino acid exchange from Arg to Gln at position 131 in the corresponding protein in case the nucleotide at position 555 is a C or in an amino acid exchange from Arg to Lys at position 131 in case the nucleotide at position 555 is an A.

The polymorphism at position 786 of SEQ ID NO: 3 consists of a replacement of the nucleotide T at this position by a C in exon 1 of UGT1A7 (which results in a Trp to Arg exchange at position 208 in the corresponding protein).

Thus, the invention relates to a method of detecting a predisposition to liver toxicity after administration of a pharmaceutically active compound based on the determination of at least one single nucleotide polymorphism, in which the single nucleotide polymorphism at position 908 in exon 5 of the UGT1 gene locus consists of the presence of a C or a G, the single nucleotide polymorphism at position 528 in exon 1 of UGT1A6 consists of the presence of a G or an A, the single nucleotide polymorphism at position 197 in exon 1 of UGT1A7 consists of the presence of a G or a C, the single nucleotide polymorphism at position 232 in exon 1 of UGT1A6 consists of the presence of a G or a T, the single nucleotide polymorphism at position 754 in exon 1 of UGT1A6 consists of the presence of an A or a G, the single nucleotide polymorphism at position 765 in exon 1 of UGT1A6 consists of the presence of an A or a C, the single nucleotide polymorphism at position 551 in exon 1 of UGT1A7 consists of the presence of an G or a T, the single nucleotide polymorphism at position 555 in exon 1 of UGT1A7 consists of the presence of an A or a C, the single nudeotide polymorphism at position 556 in exon 1 of UGT1A7 consists of the presence of an A or a C, and the single nucleotide polymorphism at position 786 in exon 1 of UGT1A7 consists of the presence of a C or a T.

A number of pharmaceutically active compounds are known which cause a hepatotoxic reaction. Examples of such compounds are nitrocatechol derivatives like entacapone, nitecapone or tolcapone. The main metabolic pathway for these drugs is glucuronidation.

Glucuronidation is an important pathway of elimination of many xenobiotics including drugs. The UGT1 enzymes are well-known to catalyze the glucuronidation of many endogeneous and exogeneous substrates, including many drugs. Pharmacokinetic experiments in human subjects have shown that the main pathway of tolcapone elimination from the body is glucuronidation (Jorga et al., Br. J. Clin. Pharmacol. (1999), 4, 513-20. Deficiencies in the elimination pathway of a drug can be the cause of adverse effects. The present invention shows a first example of genetic variations, some of which known from in vitro experiments to affect the glucuronidation activity of a number of substrates including drugs, which are significantly associated with the development of adverse effects in human patients treated with a drug. From these results it can be concluded that the method described herein can be applied to predict the predisposition to adverse effects of any drug that is metabolised by UGT1 enzymes.

Drug glucuronidation by UGTs is a major phase II conjugation reaction in the mammalian detoxification system (Burchell et al., Life Sci. (1995), 57, 1819-31). Polymorphisms in UGTs can markedly affect binding of a substrate, which can be manifested either as a clinical syndrome (if an endogenous substrate is affected) or as a change of response to a drug and/or as a adverse event (if a drug is affected). Therefore it is important to identify genetic sequence polymorphisms in the UGT1 gene in general. Nucleid acids comprising the polymorphic sequences can be used in screening assays, and for genotyping individuals. The genotyping information can be used to predict an individual's rate of metabolism for UGT1 substrates, potential drug-drug interactions, and adverse/side effects as well as diseases that result from environmental or occupational exposure to toxins. The nucleic acids can be used to establish animal, cell and in vitro models for drug metabolism. All the following identified polymorphisms are amenable to be associated with an individual's rate of metabolism for UGT1 substrates, potential drug-drug interactions, adverse/side effects and diseases that result from environmental or occupational exposure to toxins.

TABLE 1
SNP positions

| | | | SNP | | |
|---|---|---|---|---|---|
| gene | acc. no. | SNP | pos. | substit. | AA subst. |
| UGT1A6 | M84130 | exon 1 | 232 | T/G | Ser7Ala |
| | SEQ ID NO: 2 | | | | |
| | | exon 1 | 318 | C/T | silent |
| | | exon 1 | 528 | A/G | silent |
| | | exon 1 | 754 | A/G | Thr181Ala |
| | | exon 1 | 765 | A/C | Arg184Ser |
| | | exon 1 | 840 | G/T | silent |
| UGT1 A7 | U39570 | 5′UTR | 108 | T/G | |
| | SEQ ID NO: 3 | | | | |
| | | exon 1 | 197 | C/G | silent |
| | | exon 1 | 551 | T/G | Asn129Lys |
| | | exon 1 | 555 | C/A | silent or |
| | | | | | Arg131Lys |
| | | exon 1 | 556 | G/A | Arg131Gln |
| | | | | | or Lys |
| | | exon 1 | 786 | T/C | Trp208Arg |
| | | exon 1 | 920 | G/A | silent |
| | | exon 1 | 824 | C/T | silent |
| | | exon 1 | 992 | C/A | Asn/Lys |
| UGT1 A10 | U39550 | exon 1 | 959 | C/T | silent |
| | SEQ ID NO: 4 | | | | |
| UGT1 A8 | U42604 | prom | 245 | C/A | |
| | SEQ ID NO: 5 | | | | |
| UGT1 A9 | AF056188 | exon1 | 214 | C/T | silent |
| | SEQ ID NO: 6 | | | | |
| UGT ex2-5 | M84122 | intron | 117 | C/T | |
| | SEQ ID NO: 7 | | | | |
| common to | | intron | 379 | C/T | |
| all UGT1As | M84123 | exon4 | 473 | G/T | Gly/Val |
| | SEQ ID NO: 8 | | | | |
| | M84124 | 3′UTR | 423 | T/G | |
| | SEQ ID NO: 1 | | | | |
| | | 3′UTR | 780 | T/C | |
| | | 3′UTR | 908 | G/C | |
| | | 3′UTR | 1012 | C/G | |

The SNP positions in Table 1 always refer to the position in the sequence with the specified accession number in the public domain and the corresponding SEQ ID NO. given in this application. Primer sequences for genotyping assays are given in the method section. For nucleotide substitution the nucleotide of the wildtype allele is given first, same for the amino acid substitutions. SEQ ID NOs 1-3 are given above, SEQ ID NOs 4-8 are following below.

SEQ ID NO: 4 refers to Genbank accesion number U39550, which provides exon 1 of UGT1A10.

| | |
|---|---|
| 1 | CTCTCCCTCC AAGGCGAAGA CCATAATCTA CTCTTGTCTG AAATCATACA |
| 51 | AGTAGGTATC TCAGCAAATG ATACTCGTGT GTTATCGTTC TTATGAGTAA |
| 101 | ATCATTGGCA GTGAGTGTGA TTTTTTTTTT TTTTATGAAA GGATAAATAC |
| 151 | ACGCCCTCTA TTGGGGTCAG GTTTTGTGCC TGTACTTCTT CCGCCTACTG |
| 201 | TATCATAGCA GCTTAGAATC CCAGCTGCTG GCTCGGGCTG CAGTTCTCTC |
| 251 | ATCGTCGCGC AGGGTGATGG CTCGCGCAGG GTGGACCAGC CCCGTTCCTT |
| 301 | TATGTGTGTG TCTACTGCTG ACCTGTGGCT TTGCCGAGGC AGGGAAGCTG |
| 351 | CTGGTAGTGC CCATGGATGG GAGTCACTGG TTCACCATGC AGTCGGTGGT |
| 401 | GGAGAAACTT ATCCTCAGGG GGCATGAGGT GGTTGTAGTC ATGCCAGAGG |
| 451 | TGAGTTGGCA ACTGGAAAGA TCACTGAATT GCACAGTGAA GACTTACTCA |
| 501 | ACCTCGTACA CTCTGGAAGA TCAGAACCGG GAATTCATGG TTTTCGCCCA |
| 551 | TGCTCAATGG AAAGCACAGG CACAAAGTAT ATTTTCTCTA TTAATGAGTT |
| 601 | CATCCAGTGG TTTTCTTGAC TTATTTTTTT CGCATTGCAG GAGTTTGTTT |
| 651 | AATGACCGAA AATTAGTAGA ATACTTAAAG GAGAGTTCTT TTGATGCAGT |
| 701 | GTTTCTGGAT CCTTTTGATA CCTGTGGCTT AATTGTTGCT AAATATTTCT |
| 751 | CCCTCCCCTC TGTGGTCTTC ACCAGGGGAA TATTTTGCCA CCATCTTGAA |
| 801 | GAAGGTGCAC AGTGCCCTGC TCCTCTTTCC TATGTCCCCA ATGATCTCTT |
| 851 | AGGGTTCTCA GATGCCATGA CTTTCAAGGA GAGAGTATGG AACCACATCG |
| 901 | TGCACTTGGA GGACCATTTA TTTTGCCAGT ATCTTTTTAG AAATGCCCTA |
| 951 | GAAATAGCCT CTGAAATTCT CCAAACCCCT GTCACGGCAT ATGATCTCTA |
| 1001 | CAGTCACACA TCAATTTGGT TGTTGCGAAC GGACTTTGTT TTGGACTATC |
| 1051 | CCAAACCCGT GATGCCCAAC ATGATCTTCA TTGGTGGTAT CAACTGTCAT |
| 1101 | CAGGGAAAGC CATTGCCTAT GGTAAGTCAC CTCTCCTTTA GCACATTAAG |
| 1151 | AATAATCTGG CTTTGGAATT AAAAAAGGAT TCCTTACTGA ACTGTGATTT |
| 1201 | GACATTTCGT TGTGGCATTC AATTTCTTTC CAGTTTAACA AATTATTTTG |
| 1251 | TGCGAATTCA TGTACTCATC AATTATCAAA TTTTATAAAA CTGCCCTTCT |
| 1301 | TGAAAGTATA TGTAATAATT TAAAAATTAT AGATCATATT CAGGCTACAT |
| 1351 | TTTAAAATAC GATGTTTAGA AAAGTACCAA AAAACCACAG CAAGAAATGA |
| 1401 | AACTTCCGTT TTTTTGTTAT TCTATGTGAC CCCGTAGTTG AAAATGCTCT |
| 1451 | TA |

SEQ ID NO: 5 refers to Genbank accesion number U42604, which provides exon 1 of UGT1A8:

| | |
|---|---|
| 1 | GGGCATGATC TGTCCAAGGC AGAGACTATA AGCTACTCTT ATAGTACTCT |
| 51 | TATGAGATAC ATACAAGTAG GTATCTCAAA AAATGATACT CATGTATTCC |
| 101 | TGTTCTTATG AGTAAATCAT TGGCAGTGAG TGTGATTTTT TTTTTTTTTA |
| 151 | TGACAGGATC CCTACACGCC CTCTATTGGG GTCAGGTTTT GTGCCTGTAG |
| 201 | TTCTTCCGCC TACGTATCAT AGCAGTTAGA ATCCCAGCTG CTGGCTCGGG |
| 251 | CTGCAGTTCT CTCATGGCTC GCACAGGGTG GACCAGCCCC ATTCCCCTAT |
| 301 | GTGTTTCTCT GCTGCTGACC TGTGGCTTTG CTGAGGCAGG GAAGCTGCTG |
| 351 | GTAGTGCCCA TGGATGGGAG TCACTGGTTC ACCATGCAGT CGGTGGTGGA |
| 401 | GAAACTTATC CTCAGGGGGC ATGAGGTGGT TGTAGTCATG CCAGAGGTGA |
| 451 | GTTGGCAACT GGGAAAATCA CTGAATTGCA CAGTGAAGAC TTACTCAACC |
| 501 | TCATACACTC TGGAGGATCT GGACCGGGAA TTCATGGATT TCGCCGATGC |
| 551 | TCAATGGAAA GCACAAGTAC GAAGTTTGTT TTCTCTATTT CTGAGTTCAT |
| 601 | CCAATGGTTT TTTTAACTTA TTTTTTTCGC ATTGCAGGAG TTTGTTTAAT |
| 651 | GACCGAAAAT TAGTAGAATA CTTAAAGGAG AGTTCTTTTG ATGCGGTGTT |
| 701 | TCTTGATCCT TTTGATGCCT GTGCGTTAAT TGTTGCCAAA TATTTCTCCC |
| 751 | TCCCCTCTGT GGTCTTCGCC AGGGGAATAG GTTGCCACTA TCTTGAAGAA |
| 801 | GGTGCACAGT GCCCTGCTCC TCTTTCCTAT GTCCCCAGAA TTCTCTTAGG |
| 851 | GTTCTCAGAT GCCATGACTT TCAAGGAGAG AGTACGGAAC CACATCATGC |
| 901 | ACTTGGAGGA ACATTTATTT TGCCAGTATT TTTCCAAAAA TGCCCTAGAA |
| 951 | ATAGCCTCTG AAATTCTCCA AACACCTGTC ACAGCATATG ATCTCTACAG |
| 1001 | CCACACATCA ATTTGGTTGT TGCGAACAGA CTTTGTTTTG GACTATCCCA |
| 1051 | AACCCGTGAT GCCCAATATG ATCTTCATTG GTGGTATCAA CTGCCATCAG |
| 1101 | GGAAAGCCAT TGCCTATGGT AAGTCACCTC TCCTTTAGCA CATTAGGAAT |
| 1151 | AATCTTGGCT TTGGAAATTA AAAAAAGATT CCTTACTGAA TTGTGATTTG |
| 1201 | ACATTTTCAT TTGTTGCATT TCAAATTTCT TTCCAGTTTA CAGA |

SEQ ID NO: 6 refers to Genbank accesion number AF056188, which provides exon 1 of UGT1A9:

| | |
|---|---|
| 1 | CTCAGCTGCA GTTCTCTGAT GGCTTGCACA GGGTGGACCA GCCCCCTTCC |
| 51 | TCTATGTGTG TGTCTGCTGC TGACCTGTGG CTTTGCCGAG GCAGGGAAGC |
| 101 | TACTGGTAGT GCCCATGGAT GGGAGCCACT GGTTCACCAT GAGGTCGGTG |
| 151 | GTGGAGAAAC TCATTCTCAG GGGGCATGAG GTGGTTGTAG TCATGCCAGA |
| 201 | GGTGAGTTGG CAACTGGGAA GATCACTGAA TTGCACAGTG AAGACTTATT |
| 251 | CAACTTCATA TACCCTGGAG GATCTGGACC GGGAGTTCAA GGCTTTTGCC |
| 301 | CATGCTCAAT GGAAAGCACA AGTACGAAGT ATATATTCTC TATTAATGGG |
| 351 | TTCATACAAT GACATTTTTG ACTTATTTTT TTCAAATTGC AGGAGTTTGT |
| 401 | TTAAAGACAA AAAATTAGTA GAATACTTAA AGGAGAGTTC TTTTGATGCA |
| 451 | GTGTTTCTCG ATCCTTTTGA TAACTGTGGC TTAATTGTTG CCAAATATTT |
| 501 | CTCCCTCCCC TCCGTGGTCT TCGCCAGGGG AATACTTTGC CACTATCTTG |
| 551 | AAGAAGGTGC ACAGTGCCCT GCTCCTCTTT CCTATGTCCC CAGAATTCTC |
| 601 | TTAGGGTTCT CAGATGCCAT GACTTTCAAG GAGAGAGTAC GGAACCACAT |
| 651 | CATGCACTTG GAGGAACATT TATTATGCCA CCGTTTTTTC AAAAATGCCC |
| 701 | TAGAAATAGC CTCTGAAATT CTCCAAACAC CTGTTACGGA GTATGATCTC |
| 751 | TACAGCCACA CATCAATTTG GTTGTTGCGA ACGGACTTTG TTTTGGACTA |
| 801 | TCCCAAACCC GTGATGCCCA ACATGATCTT CATTGGTGGT ATCAACTGCC |
| 851 | ATCAGGGAAA GCCGTTGCCT ATGGAATTTG AAGCCTACAT TAATGCTTCT |
| 901 | GGAGAACATG GAATTGTGGT TTTCTCTTTG GGATCAATGG TCTCAGAAAT |
| 951 | TCCAGAGAAG AAAGCTATGG CAATTGCTGA TGCTTTGGGC AAAATCCCTC |
| 1001 | AGACAGTCCT GTGGCGGTAC ACTGGAACCC GACCATCGAA TCTTGCGAAC |
| 1051 | AACACGATAC TTGTTAAGTG GCTACCCCAA AACGATCTGC TTGGTCACCC |
| 1101 | GATGACCCGT GCCTTTATCA CCCATGCTGG TTCCCATGGT GTTTATGAAA |
| 1151 | GCATATGCAA TGGCGTTCCC ATGGTGATGA TGCCCTTGTT TGGTGATCAG |
| 1201 | ATGGACAATG CAAAGCGCAT GGAGACTAAG GGAGCTGGAG TGACCCTGAA |
| 1251 | TGTTCTGGAA ATGACTTCTG AAGATTTAGA AAATGCTCTA AAAGCAGTCA |
| 1301 | TCAATGACAA AAGTTACAAG GAGAACATCA TGCGCCTCTC CAGCCTTCAC |
| 1351 | AAGGACCGCC CGGTGGAGCC GCTGGACCTG GCCGTGTTCT GGGTGGAGTT |
| 1401 | TGTGATGAGG CACAAGGGCG CGCCACACCT GCGCCCCGCA GCCCACGACC |
| 1451 | TCACCTGGTA CCAGTACCAT TCCTTGGACG TGATTGGTTT CCTCTTGGCC |
| 1501 | GTCGTGCTGA CAGTGGCCTT CATCACCTTT AAATGTTGTG CTTATGGCTA |
| 1551 | CCGGAAATGC TTGGGGAAAA AAGGGCGAGT TAAGAAAGCC CACAAATCCA |
| 1601 | AGACCCATTG AGAAGTGGGT GGGAAATAAG GTAAAATTTT GAACCATTCC |
| 1651 | CTAGTCATTT CCAAACTTGA AAACAGAATC AGTGTTAAAT TCATTTTATT |
| 1701 | CTTATTAAGG AAATACTTTG CATAAATTAA TCAGCCCCAG AGTGCTTTAA |
| 1751 | AAAATTCTCT TAAATAAAAA TAATAGACTC GCTAGTCAGT AAAGATATTT |
| 1801 | GAATATGTAT CGTGCCCCCT CCGGTGTCTT TGATCAGGAT GACATGTGCC |
| 1851 | ATTTTTCAGA GGACGTGCAG ACAGGCTGGC ATTCTAGATT ACTTTTCTTA |
| 1901 | CTCTGAAACA TGGCCTGTTT GGGAGTGCGG GATTCAAAGG TGGTCCCACC |
| 1951 | GCTGCCCCTA CTGCAAATGG CAGTTTTAAT CTTATCTTTT GGCTTCTGCA |
| 2001 | GATGGTTGCA ATTGATCCTT AACCAATAAT GGTCAGTCCT CATCTCTGTC |
| 2051 | CTGCTTCATA GGTGCCACCT TGTGTGTTTA AAGAAGGGAA GCTTTGTACC |
| 2101 | TTTAGAGTGT AGGTGAAATG AATGAATGGC TTGGAGTGCA CTGAGAACAG |
| 2151 | CATATGATTT CTTGCTTTGG GGAAAAAGAA TGATGCTATG AAATTGGTGG |
| 2201 | GTGGTGTATT TGAGAAGATA ATCATTGCTT ATGTCAAATG GAGCTGAATT |
| 2251 | TGATAAAAAC CCAAAATACA GCTATGAAGT GCTGGGCAAG TTTACTTTTT |
| 2301 | TTCTGATGTT TCCTACAACT |

SEQ ID NO: 7 refers to Genbank accesion number M84122, which provides the intron of UGT1A:

| | |
|---|---|
| 1 | TTTGCATCTC AAGGATAATT CTGTAAGCAG GAACCCTTCC TCCTTTAGAA |
| 51 | GGAAGTAAAG GAGAGGAAAA TGCTGTAAAA CTTACATATT AATAATTTTT |
| 101 | TACTCTATCT CAAACACGCA TGCCTTTAAT CATAGTCTTA AGAGGAAGAT |
| 151 | ATCTAATTCA TAACTTACTG TATGTAGTCA TCAAAGAATA TGAGAAAAAA |
| 201 | TTAACTGAAA ATTTTTCTTC TGGCTCTAGG AATTTGAAGC CTACATTAAT |
| 251 | GCTTCTGGAG AACATGGAAT TGTGGTTTTC TCTTTGGGAT CAATGGTCTC |
| 301 | AGAAATTCCA GAGAAGAAAG CTATGGCAAT TGCTGATGCT TTGGGCAAAA |
| 351 | TCCCTCAGAC AGTAAGAAGA TTCTATACCA TGGCCTCATA TCTATTTTCA |
| 401 | CAGGAGCGCT AATCCCAGAC TTCCAGCTTC CAGATTAATT CTCTTAATTG |
| 451 | GAACCTTAGA TTTGGCTTTT CCCTGCCACT TCCCAACTAT TAATCCAAAG |
| 501 | GTTTTTTTTG TT |

SEQ ID NO: 8 refers to Genbank accesion number M84123, which provides exon 4 of UGT1A:

| | |
|---|---|
| 1 | AAAGATGTCC TCAAGGGACC CTGTTTTCTA GTTAGTATAG CAGATTTGTT |
| 51 | TTCTAATCAT ATTATGTCTT TCTTTACGTT CTGCTCTTTT GCCCCTCCCA |
| 101 | GGTCCTGTGG CGGTACACTG GAACCCGACC ATCGAATCTT GCGAACAACA |
| 151 | CGATACTTGT TAAGTGGCTA CCCCAAAACG ATCTGCTTGG TATGTTGGGC |
| 201 | GGATTGGATG TATAGGTCAA ACCAGGGTCA AATTAAGAAA ATGGCTTAAG |
| 251 | CACAGCTATT CTAAAGGATT GTTGAGCTTG AAAATATTAT GGCCAACATA |
| 301 | TCCTACATTG CTTTTTATCT AGTGGGGTAT CTCAACCCAC ATTTTCTTCT |
| 351 | GCAAATTTCT GCAAGGGCAT GTGAGTAACA CTGAGTCTTT GGAGTGTTTT |
| 401 | CAGAACCTAG ATGTGTCCAG CTGTGAAACT CAGAGATGTA ACTGCTGACA |
| 451 | TCCTCCCTAT TTTGCATCTC AGGTCACCCG ATGACCCGTG CCTTTATCAC |
| 501 | CCATGCTGGT TCCCATGGTG TTTATGAAAG CATATGCAAT GGCGTTCCCA |
| 551 | TGGTGATGAT GCCCTTGTTT GGTGATCAGA TGGACAATGC AAAGCGCATG |
| 601 | GAGACTAAGG GAGCTGGAGT GACCCTGAAT GTTCTGGAAA TGACTTCTGA |
| 651 | AGATTTAGAA AATGCTCTAA AAGCAGTCAT CAATGACAAA AGGTAAGAAA |
| 701 | GAAGATACAG AAGAATACTT TGGTCATGGC ATTCATGATA AAATTGTTTC |
| 751 | AAATATGAAA ACATTTACGT AGCATTTAAT ACGT |

The method in accordance with the present invention can be performed using any suitable method for detecting single nucleotide variations, such as e.g. allele specific amplification (i.e. ARMS™-allele specific amplification; ARMS referring to amplification refractory mutation system), allele specific hybridisation (ASH), oligonucleotide ligation assay (OLA) and restriction fragment length polymorphism (RFLP).

The status of a human being may be determined by reference to allelic variation at position 908 in exon 5 as defined by the position in SEQ ID NO: 1 and, if necessary, at one or more additional positions displaying a polymorphism.

The test sample of the nucleic acid carrying the said polymorphism is conveniently a sample of blood, bronchoalveolar lavage fluid, sputum, urine or other body fluid or tissue obtained from an individual. It will be appreciated that the test sample may equally be a nucleic acid sequence corresponding to the sequence in the test sample, that is to say that all or a part of the region in the sample nudeic acid may firstly be amplified using any convenient technique, e.g. polymerase chain reaction (PCR) or ligase chain reaction (LCR), before analysis of allelic variation.

It will be apparent to the person skilled in the art that there are a large number of analytical procedures which may be used to detect the presence or absence of variant nucleotides at one or more polymorphic positions of the invention. In general, the detection of allelic variation requires a mutation discrimination technique, optionally an amplification reaction and optionally a signal generation system. International patent application WO 00/06768 lists a number of amplification techniques and mutation detection techniques, some based on PCR. These may be used in combination with a number of signal generation systems, a selection of which is also listed in WO 00/06768. Many current methods for the detection of allelic variation are reviewed by Nollau et al., Clin. Chem. 43, 1114-1120, 1997; and in standard textbooks, for example “Laboratory Protocols for Mutation Detection”, Ed. by U. Landegren, Oxford University Press, 1996 and “PCR”, 2nd Edition by Newton & Graham, BIOS Scientific Publishers Limited, 1997.

The invention also relates to diagnostic nucleic acids comprising within their sequence the polymorphism at position 908 of exon 5 of UGT1 (SEQ ID NO: 1), the polymorphism at position 754 in exon 1 (SEQ ID NO: 2) or the polymorphism at position 765 of exon 1.

The term “diagnostic nucleic acid” refers to a nucleotide sequence of at least 17 nucleotides in length which corresponds to part or all of the human UGT1 gene. The diagnostic nucleic acid is preferably a part of the human UGT1 gene which part expresses the polymorphism. A length of 17 to 100 nucleotides is preferred.

Furthermore the invention relates to allele specific primers which can be used as diagnostic primers for detecting a polymorphism in the UGT1 gene capable of hybridizing to nucleic acids comprising within their sequence the polymorphisms as defined above.

An allele specific primer is used, generally together with a constant primer, in an amplification reaction such as a PCR reaction, which provides the discrimination between alleles through selective amplification of one allele at a particular sequence position e.g. as used for ARMS™ assays. The length of the allele specific primer is preferably 17-50 nucleotides, more preferably about 17-35 nucleotides, most preferably about 17-30 nucleotides.

Preferably the allele specific primer corresponds exactly with the allele to be detected but derivatives thereof are also contemplated wherein about 6-8 of the nucleotides at the 3′ terminus correspond with the allele to be detected and wherein up to 10, such as up to 8, 6, 4, 2, or 1 of the remaining nucleotides may be varied without significantly affecting the properties of the primer. Often the nucleotide at the −2 and/or −3 position (relative to the 3′ terminus) is mismatched in order to optimize differential primer binding and preferential extension from the correct allele discriminatory primer only.

Suitable examples of such diagnostic allele specific primers are the following:

| | | |
|---|---|---|
| UGT1A6 T181S: | GGTGTTCCCTGGAGCATA | (SEQ ID NO:24) |
| UGT1A6 T181S: | GGTGTTCCCTGGAGCATG | (SEQ ID NO:25) |
| UGT1A6 R184S: | GACACAGGGTCTGGGCTT | (SEQ ID NO:27) |
| UGT1A6 R184S: | GACACAGGGTCTGGGCTG | (SEQ ID NO:28) |
| UGT1A-3′ 908-2: | TGCAGTAGGGGCAGCG | (SEQ ID NO:30) |
| UGT1A-3′ 908-2 | TGCAGTAGGGGCAGCC. | (SEQ ID NO:31) |

Any convenient method of synthesis may be used to manufacture primers. Examples of such methods may be found in standard textbooks, for example “Protocols for Oligonucleotides and Analogues; Synthesis and Properties,” Methods in Molecular Biology Series; Volume 20; Ed. Sudhir Agrawal, Humana ISBN: 0-89603-247-7; 1993; 1st Edition. If required primers may be labelled to facilitate detection.

Furthermore the invention relates to allele-specific oligonucleotide probes for detecting a polymorphism in the UGT1 gene capable of hybridizing to diagnostic nucleic acids comprising within their sequence the polymorphisms as defined above.

The length of the allele-specific oligonucleotide probes are preferably 17- 50 nucleotides, more preferably about 17-35 nucleotides, most preferably about 17-30 nucleotides.

The design of such probes will be apparent to the person skilled in the art. In general such probes will comprise base sequences entirely complementary to the corresponding wild type or variant locus in the gene. However, if required one or more mismatches may be introduced, provided that the discriminatory power of the oligonucleotide probe is not unduly affected. The probes of the invention may carry one or more labels to facilitate detection.

Furthermore the invention relates to diagnostic kits comprising one or more allele-specific oligonucleotide primers or allele-specific oligonucleotide probes for detecting a polymorphism in the UGT1 gene.

The diagnostic kits may comprise appropriate packaging and instructions for use in the methods of the invention. Such kits may further comprise one or more appropriate buffers and one or more polymerases such as thermostable polymerases, for example Taq polymerase. Such kits may also comprise companion/constant primers and/or control primers or probes. A companion/constant primer is one that is part of the pair of primers used to perform PCR. Such primer usually complements the template strand precisely.

Furthermore the invention relates to a pharmaceutical pack comprising a pharmaceutically active compound like Tolcapone and instructions for administration of the drug to human beings diagnostically tested for a single nucleotide polymorphism according to a method of the present invention.

Furthermore the invention relates to a computer readable medium having stored thereon a sequence information for the polymorphism at position 908 of exon 5 of UGT1.

This invention further relates to a method for performing sequence identification, which methods comprise the steps of providing a nucleic acid sequence carrying e. g. the polymorphic site of position 908 of exon 5 or a complementary strand thereof or a fragment thereof of at least 20 bases; and comparing said nucleic acid sequence to at least one other nucleic acid or polypeptide sequence to identify identity.

The invention is further illustrated by the following figures:

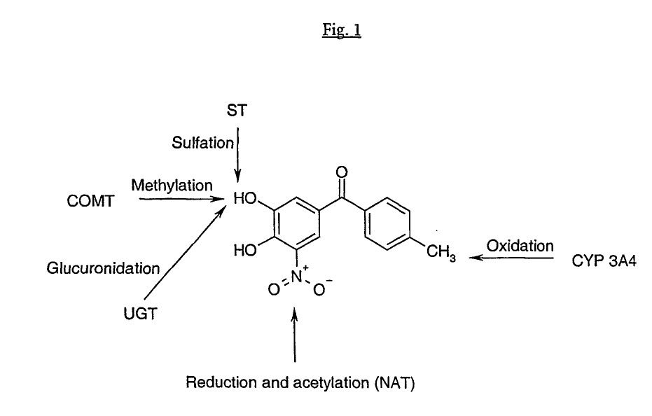

FIG. 1 shows the primary metabolic routes of tolcapone in the liver. Tolcapone is oxidized by cytochrome P450 3A4 (CYP3A4), the nitro group is reduced and acetylated by N-acetyltransferase (NAT). The phenolic hydroxy group can be sulfated by sulfotransferase (ST) or methylated by catechol-O-methyl transferase (COMT). Glucuronidation of the hydroxy group , a major reaction of detoxification in the liver, is catalyzed by UDP-glucuronosyltransferase (UGT). Subsequent oxidation or conjugation with glucuronate, sulphate and acetate further modifies primary metabolites.

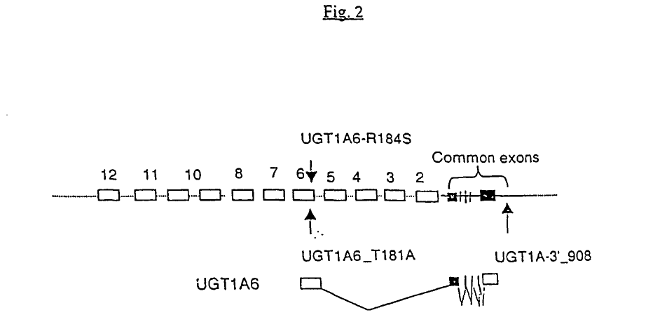

FIG. 2 represents the UGT1A gene structure. The UGT1A gene spans more than 500 kb, and consists of at least 12 promoters and first exons which can be spliced with the common exons to result in 12 different UGT1A enzymes. The structure of the UGT1A6 transcript is shown below. The arrows indicate the relative position of the polymorphic markers used in this study. UGT-3′_{—}908 represents the polymorphism at position 908 in exon 5 as defined by the position in SEQ ID NO: 1. This polymorphism in the 3′UTR (untranslated region) can potentially affect the expression of all nine functional UGT1A enzymes. The other two polymorphisms in exon 1A6 affect the protein structure of UGT1A6.

Unless otherwise defined, all technical and scientific terms used herein have the same meaning as commonly understood by the person skilled in the art to which this invention belongs.

The following examples are provided for illustration of the invention, but are not intended to limit the scope of what is regarded as the invention.

### EXAMPLES

Analysis of Tolcapone Induced Livertoxicity

Selection of Patients

The study protocol and the informed consent form were submitted for approval to the local ethical committees in the respective countries. All patients provided written informed consent for their blood sample to be used for genotypying. The consent could be withdrawn up to a month later, if the patients changed their mind.

All the samples were assigned new independent codes and a month after sample collection the link between the new and original codes was deleted. This was an added measure to ensure patient confidentiality; however, as a consequence it is not possible to retrieve genotype information based on the patient's name or number used in the original clinical trial. In approximately 15 years time, all blood and DNA samples will be destroyed.

Initially, 645 patients who had received tolcapone in previous clinical trials were considered for inclusion in this retrospective genetic analysis. This included 215 patients who had displayed liver enzyme levels of ≧1.5× the upper limit of normal (ULN) and 430 patients who had normal liver enzyme levels. Each patient with elevated liver transaminases (ELT) was matched with at least two control patients from the same study for gender and age (ethnic matching was not necessary since the great majority of the target patients were Caucasians). Disease severity had already been controlled for by the original study inclusion criteria. Of the 215 ELT patients, 135 ELT patients were enrolled in the study, 31 patients did not participate because their respective investigators could not obtain ethical approval to conduct the study, and 49 patients were either lost to follow-up or deceased. In total, 409 patients participated in this pharmacogenetic analysis. The distribution of the patients across the different sites and countries is shown in Table 2.

TABLE 2
patient distribution

| | | No of ELT | No of Controls | |
|---|---|---|---|---|
| Country | No of Sites | patients | patients | Total |
| Austria | 2 | 2 | 7 | 9 |
| Australia | 1 | 2 | 4 | 6 |
| Canada | 14 | 31 | 74 | 106 |
| Switzerland | 5 | 4 | 10 | 14 |
| Germany | 9 | 11 | 29 | 40 |
| Denmark | 4 | 8 | 13 | 21 |
| Spain | 4 | 4 | 6 | 10 |
| UK | 4 | 8 | 10 | 18 |
| Italy | 5 | 12 | 23 | 35 |
| Norway | 2 | 5 | 4 | 9 |
| USA | 20 | 48 | 94 | 142 |
| Total | 70 | 135 | 274 | 410 |

Preparation of the Samples

Single blood samples (9ml) were collected in EDTA tubes. These were frozen and stored between −20 and −70° C., before being sent to the Roche Central Sample Office (CSO) in Basel, Switzerland, where they were aliquoted into three tubes and assigned new, independent codes on bar code labels to assure patient anonymity. Two samples of blood (1 ml and 4 mls) were sent to the Roche Sample Repository (RSR) at Roche Molecular Systems (RMS) in Alameda, Calif. The remaining 4ml aliquot was stored at −80° C. in the CSO in Basel, Switzerland. All procedures performed on the samples at the RSR were done according to established standard operating procedures using GCP guidelines.

DNA was extracted from 400 μl of the whole blood using a silica gel-based extraction method (QiaAmp DNA Blood kit, Valencia, Calif.). Controls included 10 mM Tris pH 8.0, 1 mM EDTA (TE) buffer and whole blood from a blood unit with a known yield of DNA.

Samples were genotyped for eight different single nucleotide polymorphisms (SNPs) using a combination of the amplification refractory mutation system (ARMS) that relies on 3′ terminal mismatches between the PCR primers and the template being amplified according to Newton et al., Nucleic Acids Res. (1989), 17(7), 2503-16.

Analysis of any point mutation in DNA was transformed by using the amplification refractory mutation system (ARMS, Nucleic Acids Res. (1989), 17(7), 2503-16) and using the kinetic thermal cycler (KTC) format of the polymerase chain reaction. This method allows discrimination of single nudeotide polymorphisms (SNP) in a single-tube without the use of fluorescent probes (Higuchi et al., Biotechnology (1993), 11, 1026-1030).

In the KTC format, the generation of double-stranded amplification product is monitored using a DNA intercalating dye and a thermal cycler which has a fluorescence-detecting CCD camera attached (PE-Bipsystems GeneAmp 5700 Sequence Detection System). Fluorescence in each well of the PCR amplification plate is measured at each cycle of annealing and denaturation. The cycle at which the relative fluorescence reached a threshold of 0.5 using the SDS sofiware from PE-Biosystems was defined as the Cₜ.

The amplification reactions were designed to be allele-specific, so that the amplification reaction was positive if the polymorphism was present and the amplification reaction was negative if the polymorphism was absent. For each bi-allelic polymorphism, one well of the amplification plate was set up to be specific for allele 1 and a second well was set up to be specific for allele 2. For each polymorphism to be detected, three primers were designed—two allele-specific primers and one common primer (Table 3). Reactions for allele 1 contained allele 1-specific primer and the common primer and reactions for allele 2 contained allele 2-specific primer and the common primer.

TABLE 3

list of oligonucleotide primers used for polymorphism detection

| | | | | | |
|---|---|---|---|---|---|
| | | | | Primer | |
| | Primer | | SEQ ID | concentration | Annealing |
| Marker | type | Nucleotide sequence | NO | (in μM) | temperature |

| | | | | | | |
|---|---|---|---|---|---|---|
| COMT V158M | AS1 | GCACACCTTGTCCTTCAT | 9 | 0.4 | 58 | ` |
| COMT V158M | AS2 | GCACACCTTGTCCTTCAC | 10 | 0.4 | 58 | |
| COMT V158M | common | CATCACCATCGAGATCAAC | 11 | 0.4 | 58 | |
| CYP3A4 A/G | AS1 | CTATTAAATCGCCTCTCTCT | 12 | 0.4 | 56 | |
| CYP3A4 A/G | AS2 | CTATTAAATCGCCTCTCTCC | 13 | 0.4 | 56 | |
| CYP3A4 A/G | common | GGATGAATTTCAAGTATTT | 14 | 0.4 | 56 | |
| MnSOD V-9A | AS1 | AGCCCAGATACCCCAAAG | 15 | 0.4 | 58 | |
| MnSOD V-9A | AS2 | AGCCCAGATACCCCAAAA | 16 | 0.4 | 58 | |
| MnSOD V-9A | common | TGTGCTTTCTCGTCTTCA | 17 | 0.4 | 58 | |
| NAT2 I114T | AS1 | TGTAATTCCTGCCGTCAG | 18 | 0.2 | 58 | |
| NAT2 I114T | AS2 | TGTAATTCCTGCCGTCAA | 19 | 0.2 | 58 | |
| NAT2 I114T | common | ATACAGCACTGGCATGG | 20 | 0.2 | 58 | |
| SULT1A1 R213H | AS1 | CCTGGAGTTTGTGGGGCG | 21 | 0.2 | 58 | |
| SULT1A1 R213H | AS2 | CCTGGAGTTTGTGGGGCA | 22 | 0.2 | 58 | |
| SULT1A1 R213H | common | TGAACCATGAAGTCCACG | 23 | 0.2 | 58 | |
| UGT1A6 T181S | AS1 | CGTGTTCCCTGGAGCATA | 24 | 0.2 | 58 | |
| UGT1A6 T181S | AS2 | CGTGTTCCCTGGAGCATG | 25 | 0.2 | 58 | |
| UGT1A6 T181S | common | GAATGTAGGACACAGGGTCT | 26 | 0.2 | 58 | |
| UGT1A6 R184S | AS1 | GACACAGGGTCTGGGCTT | 27 | 0.2 | 58 | |
| UGT1A6 R184S | AS2 | GACACAGGGTCTGGGCTG | 28 | 0.2 | 58 | |
| UGT1A6 R184S | common | TACCTCTTCAGGGGTTTTC | 29 | 0.2 | 58 | |
| UGT1A-3′ 908-2 | AS1 | TGCAGTAGGGGCAGCG | 30 | 0.2 | 58 | |
| UGT1A-3′ 908-2 | AS2 | TGCAGTAGGGGCAGCC | 31 | 0.2 | 58 | |
| UGT1A-3′ 908-2 | common | GGAGTGCGGGATTCAA | 32 | 0.2 | 58 | |

The amplification conditions were as follows: 10 mM Tris pH 8.0, 40 mM KCl, 2 mM MgCl₂, 50 μm each of dATP, dCTP, and dGTP, 25 μm of TTP and 75 μm of dUTP, 4% DMSO, 0.2× SyBr Green (Molecular Probes, Eugene, Oreg.), 2% glycerol, uracil N-glycosylase (UNG, 2 units), Stoffel Gold DNA polymerase (15 units, for reference see Nature (1996), 381, 445-6) and primers in an 85 μl volume for each well. The concentration of the primers used for each assay are listed in Table 2.30 ng of DNA in a 15 μl volume was then added to each well.

To reduce the possibility of contamination by pre-existing amplification product, the assay procedure included the incorporation of dUTP into the amplification product and an incubation step for UNG degradation of pre-existing U-containing products (Longo et al, Gene (1990), 93,125-128).

Amplification reactions were prepared using an aliquoting robot (Packard Multiprobe II, Meriden, Conn.) in 96-well amplification plates identified by barcode labels generated by the experiment management database. Parameters for procedures performed by the robot were set to minimize the possibility of cross-contamination. For each plate of 81 samples, 5 samples were run in duplicate and the duplicate results were analysed to determine that they matched.

The thermal cycling conditions were as follows: 5 minutes at 50° C. for UNG degradation of any previously contaminating PCR products, 12 minutes at 95° C. for Stoffel Gold polyrnerase activation, 55 cycles of denaturation at 95° C. and annealing at the annealing temperature indicated in Table 2, followed by a dissociation step of 1 minute at 1 degree increments from 60° C. to 95° C. The amplification reactions were run in PE Biosystems GeneAmp 5700 Sequence Detection Systems (SDS) instruments (Foster City, Calif.). The first derivatives of the dissociation curves were produced by the SDS software and examined as needed to confirm that the fluorescence in a given reaction was due to amplification of a specific product with a well-defined dissociation peak rather than non-specific primer-dimer. Product differentiation was done by Analysis of DNA Melting Curves during PCR following the method of K. M. Ririe et al., Anal. Biochem. (1997), 245, 154-160.

The Cₜ of each amplification reaction was determined and the difference between the Cₜ for allele 1 and allele 2 (delta Cₜ) was, used as the assay result. Samples with delta Cₜs between −3.0 and 3.0 were considered heterozygous (A1/A2). Samples with delta Cₜs below −3.0 were considered homozygous for A1 (A1/A1); samples with delta Cₜs above 3.0 were considered homozygous for A2 (A2/A2). In most cases, the delta Cₜ differences between the three groups of genotypes were well-defined and samples with Cₜ values close to 3.0 were re-tested as discrepants.

Each assay was run on a panel of 14 cell line DNAs to identify cell lines with the appropriate genotypes for use as controls on each assay plate (A1/A1, A1/A2, and A2/A2). The cell line DNA was obtained from the Human Genetics Department, Roche Molecular Systems (RMS) Alameda, Calif. and was extracted using the Qiagen extraction kits (QiaAmp DNA Blood kits, Valencia, Calif.). The genotypes of the cell line DNAs were confirmed by DNA sequencing. Three cell line DNAs (A1/A1, A1/A2, and A2/A2) were run as controls on each plate of clinical trial samples and used to determine the between-plate variability. In addition, DNA from two cell lines were run in quadruplicate for each assay to determine the within-plate assay variability. The Cₜ values obtained for the control cell lines were analyzed to determine the cutoff for the delta Cₜ values obtained for the clinical trial samples.

A data file containing the Cₜ values for each well was generated by the SDS software and entered into the experiment management database. A data file with the final genotypes identified by the independent code was extracted from the database and matched to the clinical data also identified by the independent code for the statistical analysis.

For all other single nucleotide polymorphisms (SNPs) discovery and genotyping was done by double-stranded DNA sequencing using an ABI capillary sequencer and Big Dye chemistry (ABI). The primers used to amplify all exons are shown below and were also used as sequencing primers. Publicly available genomic sequences were used as references for primer design. All polymorphisms were targeted with these pairs-of-primer sets:

| | | | |
|---|---|---|---|
| UGT1A6-1 fragment: | UGT1A6-F1 | ACACGGCCATAGTTGGTTCA | (SEQ ID NO:33) |
| | UGT1A6-R1 | CAGTTGATGAAGTACAGGCC | (SEQ ID NO:34) |
| UGT1A6-2 fragment | UGT1A6-F2 | TGTAGTGGTGGTGCCTGAAG | (SEQ ID NO:35) |
| | UGT1A6-R2 | GACAGCTGATGGGAGTTCTTC | (SEQ ID NO:36) |
| UGT1A7-1 fragment: | UGT1A7-F1 | GAGGGCAGGITCTATCGTAC | (SEQ ID NO:37) |
| | UGT1A7-R1 | GGGCACTGTGCACCTTCTTC | (SEQ ID NO:38) |
| UGT1A7-2 fragment: | UGT1A7-F2 | ACGGGACGATTGGGAAGTGC | (SEQ ID NO:39) |
| | UGT1A7-R2 | ACTTAGATATCAACAAGTGCTGC | (SEQ ID NO:40) |
| UGT1A8 fragment: | UGT1A8-F | GGGCATGATCTGTCCAAGGC | (SEQ ID NO:41) |
| | UGT1A8-R | GGTTGAGTAAGTCTTCACTGTG | (SEQ ID NO:42) |
| UGTlA9 fragment: | UGT1A9-F | CTGAGGTGCAGTTCTCTG | (SEQ ID NO:43) |
| | UGT1A9-R | CCAGATGGTCGAGGGTATATG | (SEQ ID NO:44) |
| UGT1ALO fragment: | UGT1A10-F | GAGTTCATCCAGTGGTTTTC | (SEQ ID NO:45) |
| | UGT1A10-R | CAGTTCAGTAAGGAATCC | (SEQ ID NO:46) |
| UGT1in fragment: | UGT1Ain-F | CAAGGATAATTGTGTAAGGAGG | (SEQ ID NO:47) |
| | UGT1Ain-R | GGATTAATAGTTGGGAAGTGGC | (SEQ ID NO:48) |
| UGT1ex4 fragment: | UGT1ex4-F | GGCCAAGATATCCTACATTG | (SEQ ID NO:49) |
| | UGT1ex4-R | GGTATTAAATGGTACGTAAATGT | (SEQ ID NO:50) |
| UGT1-ex5-1 fragment: | UGT1ex5-1-F | GAGTTAGGGATGCTTGTGCG | (SEQ ID NO:51) |
| | UGT1ex5-1-R | GCACTCTGGGGGTGATTAAT | (SEQ ID NO:52) |
| UGT1-ex5-2 fragment: | UGT1ex5-2-F | GGTGGTGACAGTGGCGTTC | (SEQ ID NO:53) |
| | UGT1ex5-2-R | CAGTGGAGTCCAAGCCATTC | (SEQ ID NO:54) |
| UGT1-ex5-3 fragment: | UGT1ex5-3-F | GATGGTTGCAATTGATCC | (SEQ ID NO:55) |
| | UGT1ex5-3-R | TTAGITGTAGGAAAGATCAG | (SEQ ID NO:56) |

Primer UGT1A6-F1 corresponds to positions 3 to 22 in exon 1 of UGT1A6 as defined by the positions in SEQ ID NO: 2. Primer UGT1A6-R1 corresponds to the complementary strand and hybridizes to positions 571 to 590 as defined by the positions in SEQ ID NO: 2. Primer UGT1A6-F2 refers to positions 381 to 400 in exon 1 of UGT1A6 as defined by the positions in SEQ ID NO: 2. Primer UGT1A6-R2 corresponds to the complementary strand and hybridizes to positions 901 to 921 as defined by the positions in SEQ ID NO: 2.

Primer UGT1A7-F1 corresponds to positions 78 to 98 in exon 1 of UGT1A7 as defined by the positions in SEQ ID NO: 3. Primer UGT1A7-R1 corresponds to the complementary strand and hybridizes to positions 696 to 715 as defined by the positions in SEQ ID NO: 3. Primer UGT1A7-F2 corresponds to positions 459 to 478 in exon 1 of UGT1A7 as defined by the positions in SEQ ID NO: 3. Primer UGT1A7-R2 corresponds to the complementary strand and hybridizes to positions 1190 to 1212 as defined by the positions in SEQ ID NO: 3.

Primer UGT1A8-F corresponds to positions 1 to 20 in exon 1 of UGT1A8 as defined by the positions in SEQ ID NO: 5. Primer UGT1A8-R hybridizes to positions 479 to 500 as defined by the positions in SEQ ID NO: 5.

Primer UGT1A9-F corresponds to positions 1 to 18 in exon 1 of UGT1A9 as defined by the positions in SEQ ID NO: 6. Primer UGT1A9-R hybridizes to positions 257 to 277 as defined by the positions in SEQ ID NO: 6.

Primer UGT1A10-F corresponds to positions 596 to 615 in exon 1 of UGT1A7 as defined by the positions in SEQ ID NO: 4. Primer UGT1A10-R hybridizes to positions 1177 to 1194 as defined by the positions in SEQ ID NO: 4.

Primer UGT1Ain-F corresponds to positions 10 to 31 in the intron of UGT1A as defined by the positions in SEQ ID NO: 7. Primer UGT1Ain-R hybridizes to positions 475 to 496 as defined by the positions in SEQ ID NO: 7.

Primer UGT1ex4-F corresponds to positions 291 to 310 in exon 4 of UGT1A as defined by the positions in SEQ ID NO: 8. Primer UGT1ex4-R hybridizes to positions 761 to 784 as defined by the positions in SEQ ID NO: 8.

Primer UGT1ex5-1-F corresponds to positions 63 to 82 in exon 5 of UGT1 as defined by the positions in SEQ ID NO: 1. Primer UGT1ex5-1-R hybridizes to positions 684 to 703 as defined by the positions in SEQ ID NO: 1. Primer UGT1ex5-2-F corresponds to positions 461 to 480 in exon 5 of UGT1 as defined by the positions in SEQ ID NO: 1. Primer UGT1ex5-2-R hybridizes to positions 1082 to 1101 as defined by the positions in SEQ ID NO: 1. Primer UGT1ex5-3-F corresponds to positions 959 to 976 in exon 5 of UGT1 as defined by the positions in SEQ ID NO: 1 and primer UGT1ex5-3-R hybridizes to positions 1261 to 1280 as defined by the positions in SEQ ID NO: 1.

Fourty nanograms of genomic DNA were PCR-amplified in 50 μl reactions using an automated PCR machine. Reaction conditions varied as follows. For the amplification of the UGT1A6-fragment conditions were as follows: 10 mM Tris pH 8.3, 50 mM KCl, 1.5 mM MgCl₂, 0.2 mM of each dNTP, 0.4 μM of each primer and 1.5U Boehringer Taq Polymerase. The thermocycling protocol consisted of an initial incubation of 95° C. for 15 min. followed by 35 cycles of 94° C. for 1 min., 57° C. for 30 sec., 72° C. for 1 min., and one final extension step of 72° C. for 10 min. The UGT1A7-1 fragment was amplified using Qiagen PCR buffer with 1.5 mM MgCl₂, 0.2 mM of each dNTP, 0.4 μM of each primer and 1.5U Boehringer Taq Polymerase. The thermocycling protocol was the same as for UGT1A6-fragment with one exception: the annealing temperature was 61° C. For UGT1A7-2 fragment PCR conditions were as follows: 150 mM Tris pH 8.5, 15 mM (NH₄)₂SO₄, 3.5 mM MgCl₂, 0.2 mM of each dNTP, 0.4 μM of each primer and 1.5U Qiagen Hot Start Taq Polymerase. Thermocycling was done using a touch-down PCR protocol. After an iniatial amplification of 95° C. for 10 min. followed 5 cycles of 95° C. for 1 min., 62° C. for 30 sec. (minus 0.5° C. per cycle), 72° C. for 1 min and thirty cycles of 95° C. for 1 min., 60° C. for 30 sec., 72° C. for 1 min. and a final extension step of 72° C. for 10 min. After PCR amplification fragments were purified using the Qiaquick PCR purification kit on a Biorobot 9600. Cycle sequencing was performed on an automated PCR machine using ABI Big Dye terminator chemistry according to the manufacturer's intruction with the following changes: 2.5-5 ng/100 bp of PCR product were mixed with 2 μl Big Dye terminatior mix, oligonucleotide primer concentration was 10 pmol, if necessary 5% DMSO was added to the reaction; the final reaction volume was 10 μl. Sequencing reactions were subjected to 28 cycles at 93° C. for 30 sec, 48° C. for 30 sec, and 58° C. for 120 sec., followed by an ethanol/NaOAc precipitation. After decanting the ethanol, samples were evaporated to dryness using a SpeedVac for 2 min. and were resuspended in 45 μl ultrapure water (MERCK, HPLC grade). 2.5 ml were loaded on an ABI 3700 capillary sequencer using POP5 as a polymer. After sequencing, the polymorphism analyses were done using Polyphred software (licenced from University of Washington).

Selection and Discovery of Genetic Markers

The genetic markers were selected based on the known pharmacology of tolcapone and knowledge from the literature of genetic polymorphisms that could affect the activity of corresponding and relevant gene products. The main metabolic pathway for tolcapone elimination is glucuronidation by UGT1 enzymes.

In addition to polymorphisms in the UGT1 enzyme, genetic polymorphisms in genes encoding the following enzymes involved in tolcapone metabolism (FIG. 1) were selected: catechol-O-methyl transferase (COMT) according to Lachman et al., Pharmacogenetics (1996), 6, 243-250; N-acetyl transferase (NAT2, for reference see Vatsis et al., Proc. Natl. Acad. Sci. (1991), 88, 6333-6337; Bandmann et al., Lancet (1997), 350, 1136-1139); liver sulfotransferase (SULTlAl) according to Ozawa et al., Chem. Biol. Interact. (1998), 109, 237-248 and Cytochrome P450 enzyme (CYP3A4, Rebbeck et al., J. Natl. Cancer Inst. (1998), 50, 1225-1229). The genotype for manganese superoxide dismutase (MnSOD, Shimoda-Matsubayashi et al., Biochem. Biophys. Res. Commun. (1996), 226, 561-565), involved in oxidative stress response, was also investigated.

UDP-glucuronosyltransferase 1A6 (UGT1A6) was selected as it potentially metabolises tolcapone via glucuronidation. The alleles Thr181Ala and Arg184Ser are described as showing reduced activity for levodopa and other substrates (Ciotti et al., Pharmacogenetics (1997), 7, 485-495).The known genetic polymorphisms in the UGT1A gene affect only single members of this gene cluster of twelve genes (FIG. 2). Therefore, it was reasoned that genetic variations in the potentially common regulatory region, that is the 3′-end of the gene, could have an effect on the expression of any of the twelve UGT1A genes. Moreover, it was suspected that UGT1A7 may be involved in the elimination of tolcapone. In order to identify new genetic polymorphisms common exons 2-5 and the 3′ untranslated region of UGT1A and exon 1 of the UGT1A6, UGT1A7, UGT1A8, UGT1A9 and UGT1A10 genes were sequenced in 47 different DNA samples from ethnically diverse individuals. The 300-700 bp fragments were column purified with the Qiaquick PCR purification kit on a Biorobot 9600 and both strands were sequenced on an ABI3700 capillary sequencer using dye-terminator chemistry and the PCR amplification primers as sequencing primers as described in detail above. A G/C variation was identified designated as UGT1A-3′_{—}908, which occurred in the following frequencies: CC: 0.63; GC:0.33; GG:0.04. The number 908 refers to the position of the SNP relative to the DNA sequence with Genbank accession number M84124 from the public data bases. Moreover, the following polymorphisms have been identified in UGT1A6, UGT1A7, UGT1A8, UGT1A9 and UGT1A10 genes:

UGT1A6exon1_{—}318 and UGT1A6exon1-528. The number refers to the position of the SNP relative to the DNA sequence with Genbank accession number M84130 from the public database.

UGT1A7 exon1_{—}197, UGT1A7exon1_{—}824, UGT1A7exon1_{—}920, and UGT1A7exon1_{—}992. The numbers refer to the position of the SNP relative to the DNA sequence with Genbank accession number U39570 from the public database.

UGT1A8promoter_{—}245. The number refers to the position of the SNP relative to the DNA sequence with Genbank accession number U42604 from the public database.

UGT1A9exon1_{—}214. The number refers to the position of the SNP relative to the DNA sequence with Genbank accession number AF056188 from the public database.

UGT1A10exon1_{—}959. The number refers to the position of the SNP relative to the DNA sequence with Genbank accession number U39550 from the public database.

UGT1Aintron_{—}117 and UGT1Aintron_{—}379. The numbers refer to the position of the SNPs relative to the DNA sequence with Genbank accession number M84122 from the public database.

UGT1Aexon4_{—}473. The number refers to the position of the SNP relative to the DNA sequence with Genbank accession number M84123 from the public database.

UGT1Aexon5_{—}423, UGT1Aexon5_{—}780, UGT1Aexon5_{—}908 (described above in detail) and UGT1Aexon5_{—}1012. The numbers refer to the position of the SNPs relative to the DNA sequence with Genbank accession number M84124 from the public domain.

The patient samples were divided into two groups. Group 1 contained samples from case patients whose aspartate aminotransferase (AST:SGOT), alanine aminotransferse (ALT:SGPT), or bilirubin values were ≧1.5×ULN of the investigators range while taking tolcapone treatment. Group 2 contained samples from control patients whose SGOT, SGPT, and bilirubin values were below 1×ULN when measured while taking tolcapone treatment.

For each genotype, the following analyses were performed:

a) Analysis of the entire genotype: patients were classified according to the following three categories: homozygous 1/1 (two copies of allele 1 and no copies of allele 2), heterozygous 1/2 (one copy of allele 1 and one copy of allele 2) or homozygous 2/2 (no copies of allele 1 and two copies of allele 2). Analysis was conducted to assess whether there were differences in the proportion of case patients in each of these groups, compared with the proportion of control patients. The Cochran-Maentel-Hanszel (CMH) test was applied to the data presented in a 2-by-3 table (the two columns indicating presence or absence of liver function abnormality and the three rows indicating the three categories of the genotype).

b) Analysis of alleles: the presence of allele 1 or 2 in case patients was compared to the presence of the respective allele in the control patients. For each allele, the CMH test was applied using a 2-by-2 table (the two columns indicating presence or absence of liver function abnormality and the two rows indicating the presence or absence of the respective allele), and the case-control odds ratio and 95% confidence interval were calculated. An odds-ratio of greater than 1.0, together with a confidence interval that does not include 1.0, indicated a positive association between the presence of the allele and the occurrence of liver function abnormality.

c) Analysis of allele counts: this analysis was conducted to compare the distribution of the alleles in patients with liver function abnormality to that of patients without abnormality. The total number of copies of allele 1 among patients with liver function abnormality was compared with the total number of copies of allele 2 among these case patients. The CMH test was applied to the data using a 2-by-2 table (the two columns indicating presence or absence of liver function abnormality and the two rows indicating the two allele counts). Again case-control odds ratio and 95% confidence intervals were obtained.

Results

A total of 409 patients treated with tolcapone, of which 135 had liver enzyme elevation of 1.5 times or more above the upper limit and 274 were matched controls, were genotyped for different genetic markers from genes encoding enzymes involved in the metabolism of tolcapone, including the UGT1 genes. The results from the analysis of the genetic markers that resulted in a significant association are presented in tables 4 to 13. All markers showing significant association to elevated liver transaminases corresponded to SNPs in the UGT1 genes.

TABLE 4
UGT1A6_765 Arg184Ser

| | | Liver Function Abnormality | |
|---|---|---|---|
| | | Absent | Present |
| Genotype | Homozygous Arg/Arg | 128 (46.7%) | 45 (33.3%) |
| | Heterozygous Arg/Ser | 122 (44.5%) | 64 (47.4%) |
| | Homozygous Ser/Ser | 24 (8.8%) | 26 (19.3%) |
| | P-value | 0.0023 | |
| Allele Arg | Absent | 24 (8.8%) | 26 (19.3%) |
| | Present | 250 (91.2%) | 109 (80.7%) |
| | Relative Risk | 0.58 | |
| | Odds Ratio | 0.40 | |
| | 95% CI | (0.22, 0.72) | |
| | P-value | 0.0023 | |
| Allele Ser | Absent | 128 (46.7%) | 45 (33.3%) |
| | Present | 146 (53.3%) | 90 (66.7%) |
| | Relative Risk | 1.47 | |
| | Odds Ratio | 1.75 | |
| | 95% CI | (1.14, 2.69) | |
| | P-value | 0.0101 | |
| Count | Allele Arg | 378 (69.0%) | 154 (57.0%) |
| | Allele Ser | 170 (31.0%) | 116 (43.0%) |

| | |
|---|---|
| Odds Ratio | 1.67 |
| 95% CI | (1.24, 2.26) |
| P-value | 0.0008 |

TABLE 5
UGT1A6_754 Thr181Ala

| | | Liver Function Abnormality | |
|---|---|---|---|
| | | Absent | Present |
| Genotype | Homozygous Thr/Thr | 144 (52.6%) | 51 (37.8%) |
| | Heterozygous Thr/Ala | 108 (39.4%) | 59 (43.7%) |
| | Homozygous Ala/Ala | 22 (8.8%) | 25 (18.5%) |
| | P-value | 0.0014 | |
| Allele Thr | Absent | 22 (8.0%) | 25 (18.5%) |
| | Present | 252 (92.0%) | 110 (81.5%) |
| | Relative Risk | 0.57 | |
| | Odds Ratio | 0.38 | |
| | 95% CI | (0.21, 0.70) | |
| | P-value | 0.0018 | |
| Allele Ala | Absent | 144 (52.6%) | 51 (37.8%) |
| | Present | 130 (47.4%) | 84 (62.2%) |
| | Relative Risk | 1.50 | |
| | Odds Ratio | 1.82 | |
| | 95% CI | (1.20, 2.77) | |
| | P-value | 0.0050 | |
| Count | Allele Thr | 396 (72.3%) | 161 (59.6%) |
| | Allele Ala | 152 (27.7%) | 109 (40.4%) |

| | |
|---|---|
| Odds Ratio | 1.76 |
| 95% CI | (1.24, 2.26) |
| P-value | 0.0008 |

TABLE 6
UGT1A-3′_908

| | | Liver Function Abnormality | |
|---|---|---|---|
| | | Absent | Present |
| Genotype | Homozygous C/C | 163 (59.5%) | 101 (74.8%) |
| | Heterozygous G/C | 97 (35.4%) | 30 (22.2%) |
| | Homozygous G/G | 14 (5.1%) | 4 (3.0%) |
| | P-value | 0.0097 | |
| Allele C | Absent | 14 (5.1%) | 4 (3.0%) |
| | Present | 260 (94.9%) | 131 (97.0%) |
| | Relative Risk | 1.51 | |
| | Odds Ratio | 1.76 | |
| | 95% CI | (0.58, 5.40) | |
| | P-value | 0.3202 | |
| Allele G | Absent | 163 (59.5%) | 101 (74.8%) |
| | Present | 111 (40.5%) | 34 (25.2%) |
| | Relative Risk | 0.61 | |
| | Odds Ratio | 0.49 | |
| | 95% CI | (0.31, 0.78) | |
| | P-value | 0.0023 | |
| Count | Allele C | 423 (77.2%) | 232 (85.9%) |
| | Allele G | 125 (22.8%) | 38 (14.1%) |

| | |
|---|---|
| Odds Ratio | 0.55 |
| 95% CI | (0.37, 0.82) |
| P-value | 0.0033 |

TABLE 7
UGT1A6_232 Ser7Ala

| | | Liver Function Abnormality | |
|---|---|---|---|
| | | Absent | Present |
| Genotype | Homozygous T/T | 112 (42.1%) | 40 (30.5%) |
| | Heterozygous T/G | 123 (46.2%) | 63 (48.1%) |
| | Homozygous G/G | 31 (11.7%) | 28 (21.4%) |
| | P-value | 0.0130 | |
| Allele T | Absent | 31 (11.7%) | 28 (21.4%) |
| | Present | 235 (88.3%) | 103 (78.6%) |
| | Relative Risk | 0.64 | |
| | Odds Ratio | 0.49 | |
| | 95% CI | (0.28, 0.84) | |
| | P-value | 0.0106 | |
| Allele G | Absent | 112 (42.1%) | 40 (30.5%) |
| | Present | 154 (57.9%) | 91 (69.5%) |
| | Relative Risk | 1.41 | |
| | Odds Ratio | 1.65 | |
| | 95% CI | (1.06, 2.58) | |
| | P-value | 0.0259 | |
| Count | Allele T | 347 (65.2%) | 143 (54.6%) |
| | Allele G | 185 (34.8%) | 119 (45.4%) |

| | |
|---|---|
| Odds Ratio | 1.56 |
| 95% CI | (1.16, 2.11) |
| P-value | 0.0037 |

TABLE 8
UGT1A6_528 A/G

| | | Liver Function Abnormality | |
|---|---|---|---|
| | | Absent | Present |
| Genotype | Homozygous A/A | 129 (48.7%) | 44 (33.8%) |
| | Heterozygous A/G | 114 (43.0%) | 60 (46.2%) |
| | Homozygous G/G | 22 (8.3%) | 26 (20.0%) |
| | P-value | 0.0008 | |
| Allele A | Absent | 22 (8.3%) | 26 (20.0%) |
| | Present | 243 (91.7%) | 104 (80.0%) |
| | Relative Risk | 0.55 | |
| | Odds Ratio | 0.36 | |
| | 95% CI | (0.20, 0.66) | |
| | P-value | 0.0106 | |
| Allele G | Absent | 129 (48.7%) | 44 (33.8%) |
| | Present | 136 (51.3%) | 86 (66.2%) |
| | Relative Risk | 1.52 | |
| | Odds Ratio | 1.85 | |
| | 95% CI | (1.20, 2.68) | |
| | P-value | 0.0053 | |
| Count | Allele A | 372 (70.2%) | 148 (56.9%) |
| | Allele G | 158 (29.8%) | 112 (43.1%) |

| | |
|---|---|
| Odds Ratio | 1.78 |
| 95% CI | (0.31, 2.42) |
| P-value | 0.0002 |

TABLE 9
UGT1A7_197 C/G

| | | Liver Function Abnormality | |
|---|---|---|---|
| | | Absent | Present |
| Genotype | Homozygous A/A | 32 (11.9%) | 25 (18.8%) |
| | Heterozygous A/C | 112 (41.6%) | 62 (46.6%) |
| | Homozygous C/C | 125 (46.5%) | 46 (34.6%) |
| | P-value | 0.0400 | |
| Allele A | Absent | 125 (46.5%) | 46 (34.6%) |
| | Present | 144 (53.5%) | 87 (65.4%) |
| | Relative Risk | 1.40 | |
| | Odds Ratio | 1.64 | |
| | 95% CI | (1.07, 2.52) | |
| | P-value | 0.0235 | |
| Allele C | Absent | 32 (11.9%) | 25 (18.8%) |
| | Present | 237 (88.1%) | 108 (81.2%) |
| | Relative Risk | 0.71 | |
| | Odds Ratio | 0.58 | |
| | 95% CI | (0.33, 1.03) | |
| | P-value | 0.0623 | |
| Count | Allele A | 176 (32.7%) | 112 (42.1%) |
| | Allele G | 362 (67.3%) | 154 (57.9) |

| | |
|---|---|
| Odds Ratio | 0.67 |
| 95% CI | (0.49, 0.90) |
| P-value | 0.0090 |

TABLE 10
UGT1A7_551 Asn129Lys

| | | Liver Function Abnormality | |
|---|---|---|---|
| | | Absent | Present |
| Genotype | Homozygous G/G | 96 (35.7%) | 59 (44.7%) |
| | Heterozygous G/T | 124 (46.1%) | 59 (44.7%) |
| | Homozygous T/T | 49 (18.2%) | 14 (10.6%) |
| | P-value | 0.0762 | |
| Allele G | Absent | 49 (18.2%) | 14 (10.6%) |
| | Present | 220 (81.8%) | 118 (89.4%) |
| | Relative Risk | 1.57 | |
| | Odds Ratio | 1.88 | |
| | 95% CI | (1.00, 3.52) | |
| | P-value | 0.0494 | |
| Allele T | Absent | 96 (35.7%) | 59 (44.7%) |
| | Present | 173 (64.3%) | 7 (55.3%) |
| | Relative Risk | 0.78 | |
| | Odds Ratio | 0.69 | |
| | 95% CI | (0.45, 1.05) | |
| | P-value | 0.0821 | |
| Count | Allele G | 116 (58.7%) | 177 (67.0%) |
| | Allele T | 222 (41.3%) | 87 (33.0%) |

| | |
|---|---|
| Odds Ratio | 0.70 |
| 95% CI | (0.51, 0.95) |
| P-value | 0.0232 |

TABLE 11
UGT1A7_555 silent or Arg131Lys

| | | Liver Function Abnormality | |
|---|---|---|---|
| | | Absent | Present |
| Genotype | Homozygous A/A | 96 (35.7%) | 58 (43.9%) |
| | Heterozygous A/C | 124 (46.1%) | 60 (45.5%) |
| | Homozygous G/G | 49 (18.2%) | 14 (10.6%) |
| | P-value | 0.0894 | |
| Allele A | Absent | 49 (18.2%) | 14 (10.6%) |
| | Present | 220 (81.8%) | 118 (89.4%) |
| | Relative Risk | 1.57 | |
| | Odds Ratio | 1.88 | |
| | 95% CI | (1.00, 3.52) | |
| | P-value | 0.0494 | |
| Allele C | Absent | 96 (35.7%) | 58 (43.9%) |
| | Present | 173 (64.3%) | 74 (56.1%) |
| | Relative Risk | 0.80 | |
| | Odds Ratio | 0.71 | |
| | 95% CI | (0.46, 1.08) | |
| | P-value | 0.1108 | |
| Count | Allele A | 316 (58.7%) | 176 (66.7%) |
| | Allele C | 222 (41.3%) | 88 (33.3%) |

| | |
|---|---|
| Odds Ratio | 0.71 |
| 95% CI | (0.52, 0.97) |
| P-value | 0.0303 |

TABLE 12
UGT1A7_556 Arg131Lys or Gln

| | | Liver Function Abnormality | |
|---|---|---|---|
| | | Absent | Present |
| Genotype | Homozygous A/A | 96 (35.8%) | 44 (33.8%) |
| | Heterozygous A/G | 123 (45.9%) | 59 (44.7%) |
| | Homozygous G/G | 49 (18.3%) | 14 (10.6%) |
| | P-value | 0.0772 | |
| Allele A | Absent | 49 (18.3%) | 14 (10.6%) |
| | Present | 219 (81.7%) | 118 (89.4%) |
| | Relative Risk | 1.58 | |
| | Odds Ratio | 1.89 | |
| | 95% CI | (1.01, 3.53) | |
| | P-value | 0.0477 | |
| Allele G | Absent | 96 (35.8%) | 59 (44.7%) |
| | Present | 172 (64.2%) | 73 (55.3%) |
| | Relative Risk | 0.78 | |
| | Odds Ratio | 0.69 | |
| | 95% CI | (0.45, 1.06) | |
| | P-value | 0.0870 | |
| Count | Allele A | 315 (58.8%) | 177 (67.0%) |
| | Allele G | 221 (41.2%) | 87 (33.0%) |

| | |
|---|---|
| Odds Ratio | 0.70 |
| 95% CI | (0.51, 0.95) |
| P-value | 0.0238 |

TABLE 13
UGT1A7_786 Trp208Arg

Liver Function Abnormality

| | | Absent | Present |
|---|---|---|---|
| Genotype | Homozygous T/T | 124 (46.3%) | 46 (34.3%) |
| | Heterozygous T/C | 114 (42.5%) | 62 (46.3%) |
| | Homozygous C/C | 30 (11.2%) | 26 (19.4%) |

| | |
|---|---|
| P-value | 0.0224 |

| | | | |
|---|---|---|---|
| Allele T | Absent | 30 (11.2%) | 26 (19.4%) |
| | Present | 238 (88.8%) | 108 (80.6%) |

| | |
|---|---|
| Relative Risk | 0.67 |
| Odds Ratio | 0.52 |
| 95% CI | (0.30, 0.92) |
| P-value | 0.0252 |

| | | | |
|---|---|---|---|
| Allele G | Absent | 124 (46.3%) | 46 (34.3%) |
| | Present | 144 (53.7%) | 86 (65.7%) |

| | |
|---|---|
| Relative Risk | 1.40 |
| Odds Ratio | 1.65 |
| 95% CI | (1.07, 2.53) |
| P-value | 0.0225 |

| | | | |
|---|---|---|---|
| Count | Allele T | 362 (67.5%) | 154 (57.5%) |
| | Allele C | 174 (32.5%) | 114 (42.5%) |

| | |
|---|---|
| Odds Ratio | 1.54 |
| 95% CI | (0.14, 2.08) |
| P-value | 0.0050 |

The main metabolic pathway for tolcapone elimination is glucuronidation. The results from the current retrospective analysis have shown a significant association between three genetic polymorphisms in the UDP-glucuronosyltransferase gene and liver function abnormality. These findings support the hypothesis that impaired elimination of tolcapone may be a cause for liver toxicity. In vitrq studies in rat hepatotocyte cultures have shown that inhibition of glucuronidation and oxidation increase cytotoxicity of tolcapone. Moreover, the UGT1A6 Ala181/Ser184 variant was shown to have reduced activity in vitro compared with the Thr181/Arg184 variant (Ciotti et. al., Pharmacogenetics 1997, 7, 485-495). This concurs with the findings from the current analysis whereby the presence of Ala181 and the absence of Ser184 were associated with an incrementally higher risk of liver abnormality. The polymorphism located in the 3′UTR of the UGT1A gene (FIG. 2) may affect the expression of all UGT1A genes involved in metabolism of Tolcapone. Alternatively, the polymorphism may be in linkage disequilibrium with another mutation that affects either the structure of the UGT1A proteins, or the expression of the gene.

No significant association was found with the other markers tested. These results do not rule out the potential contribution of other polymorphisms within the gene tested.

The relatively low odd ratios that result from these associations, are due to the multifactorial nature of drug induced liver toxicity. It was clear throughout this study that the occurrence of liver enzyme elevation upon treatment with tolcapone was the result of multiple factors including external influences such as co-medication, and or the combination of several genetic factors in different individuals.

1. A method for detecting a predisposition to a hepatototoxic reaction of a human being caused by the administration of a pharmaceutically active compound based on the determination of at least one single nucleotide polymorphism in the UDP-glucuronosyltransferase (UGT1) gene in a sample of said human being, which method comprises
determining the nucleotide at position 908 in exon 5 of the UGT1 gene as defined by the position in SEQ ID NO: 1, and/or
determining the nucleotide at position 528 in exon 1 of the UGT1A6 gene as defined by the position in SEQ ID NO: 2, and/or
determining the nucleotide at position 197 in exon 1 of the UGT1A7 gene as defined by the position in SEQ ID NO: 3, and
determining the status of the human being.

2. A method for detecting a predisposition to a hepatototoxic reaction of a human being caused by the administration of a pharmaceutically active compound based on the determination of at least one single nucleotide polymorphism in the UDP-glucuronosyltransferase (UGT1) gene in a sample of said human being, which method comprises determining the nucleotide at position 908 in exon 5 of the UGT1 gene as defined by the position in SEQ ID NO: 1 and determining the status of the human being.

3. The method according to any one of claims 1 or 2, wherein additionally the polymorphism at one or more of the following positions is determined:
position 232 in exon 1 of UGT1A6 as defined by the position in SEQ ID NO: 2, or
position 754 in exon 1 of UGT1A6 as defined by the position in SEQ ID NO: 2, or
position 765 in exon 1 of UGT1A6 as defined by the position in SEQ ID NO: 2, or
position 551 in exon 1 of UGT1A7 as defined by the position in SEQ ID NO: 3, or
position 555 in exon 1 of UGT1A7 as defined by the position in SEQ ID NO: 3, or
position 556 in exon 1 of UGT1A7 as defined by the position in SEQ ID NO: 3, or
position 786 in exon 1 of UGT1A7 as defined by the position in SEQ ID NO: 3.

4. A method according to any one of claims 1 to 3, in which the single nucleotide polymorphism at position 908 in exon 5 of the UGT1 gene locus consists of the presence of a C or a G, the single nucleotide polymorphism at position 528 in exon 1 of UGT1A6 consists of the presence of a G or an A, the single nucleotide polymorphism at position 197 in exon 1 of UGT1A7 consists of the presence of a G or a C, the single nucleotide polymorphism at position 232 in exon 1 of UGT1A6 consists of the presence of a G or a T, the single nucleotide polymorphism at position 754 in exon 1 of UGT1A6 consists of the presence of an A or a G, the single nucleotide polymorphism at position 765 in exon 1 of UGT1A6 consists of the presence of an A or a C, the single nucleotide polymorphism at position 551 in exon 1 of UGT1A7 consists of the presence of an G or a T, the single nucleotide polymorphism at position 555 in exon 1 of UGT1A7 consists of the presence of an A or a C, the single nucleotide polymorphism at position 556 in exon 1 of UGT1A7 consists of the presence of an A or a C, and the single nudeotide polymorphism at position 786 in exon 1 of UGT1A7 consists of the presence of a C or a T.

5. A method as claimed in claims 1 to 4, wherein the region containing the potential polymorphism is amplified, preferably by polymerase chain reaction, prior to determining the sequence.

6. A diagnostic nucleic acid comprising the following polymorphism containing sequences:
the nucleic acid sequence of SEQ ID NO: 1 with C at position 908 in exon 5 of UGT1 as defined by the position in SEQ ID NO: 1;
the nucleic acid sequence of SEQ ID NO: 1 with G at position 908 in exon 5 of UGT1 as defined by the position in SEQ ID NO: 1;
the nucleic acid sequence of SEQ ID NO: 2 with G at position 232 in exon 1 of UGT1A6 as defined by the position in SEQ ID NO: 2;
the nucleic acid sequence of SEQ ID NO: 2 with T at position 232 in exon 1 of UGT1A6 as defined by the position in SEQ ID NO: 2;
the nucleic acid sequence of SEQ ID NO: 2 with G at position 528 in exon 1 of UGT1A6 as defined by the position in SEQ ID NO: 2;
the nucleic acid sequence of SEQ ID NO: 2 with A at position 528 in exon 1 of UGT1A6 as defined by the position in SEQ ID NO: 2;
the nucleic acid sequence of SEQ ID NO: 2 with G at position 754 in exon 1 of UGT1A6 as defined by the position in SEQ ID NO: 2;
the nucleic acid sequence of SEQ ID NO: 2 with A at position 754 in exon 1 of UGT1A6 as defined by the position in SEQ ID NO: 2;
the nucleic acid sequence of SEQ ID NO: 2 with C at position 765 in exon 1 of UGT1A6 as defined by the position in SEQ ID NO: 2;
the nucleic acid sequence of SEQ ID NO: 2 with A at position 765 in exon 1 of UGT1A6 as defined by the position in SEQ ID NO: 2;
the nucleic acid sequence of SEQ ID NO: 3 with G at position 197 in exon 1 of UGT1A7 as defined by the position in SEQ ID NO: 3;
the nucleic acid sequence of SEQ ID NO: 3 with C at position 197 in exon 1 of UGT1A7 as defined by the position in SEQ ID NO: 3;
the nucleic acid sequence of SEQ ID NO: 3 with G at position 551 in exon 1 of UGT1A7 as defined by the position in SEQ ID NO: 3;
the nucleic acid sequence of SEQ ID NO: 3 with T at position 551 in exon 1 of UGT1A7 as defined by the position in SEQ ID NO: 3;
the nucleic acid sequence of SEQ ID NO: 3 with A at position 555 in exon 1 of UGT1A7 as defined by the position in SEQ ID NO: 3;
the nucleic acid sequence of SEQ ID NO: 3 with C at position 555 in exon 1 of UGT1A7 as defined by the position in SEQ ID NO: 3;
the nucleic acid sequence of SEQ ID NO: 3 with A at position 556 in exon 1 of UGT1A7 as defined by the position in SEQ ID NO: 3;
the nucleic acid sequence of SEQ ID NO: 3 with G at position 556 in exon 1 of UGT1A7 as defined by the position in SEQ ID NO: 3;
the nucleic acid sequence of SEQ ID NO: 3 with C at position 786 in exon 1 of UGT1A7 as defined by the position in SEQ ID NO: 3;
the nucleic acid sequence of SEQ ID NO: 3 with T at position 786 in exon 1 of UGT1A7 as defined by the position in SEQ ID NO: 3; or
a complementary strand thereof or a fragment thereof of at least 20 bases comprising at least one of the polymorphisms.

7. A diagnostic nucleic acid selected from the group consisting of the nucleic acid sequence of SEQ ID NO: 1 with C at position 908 in exon 5 as defined by the position in SEQ ID NO: 1;
the nucleic acid sequence of SEQ ID NO: 2 with G at position 528 in exon 1 of UGT1A6 as defined by the position in SEQ ID NO: 2; or
the nucleic acid sequence of SEQ ID NO: 3 with G at position 197 in exon 1 of UGT1A7 as defined by the position in SEQ ID NO: 3.

8. A diagnostic nucleic acid selected from the group consisting of the nucleic acid sequence of SEQ ID NO: 1 with G at position 908 in exon 5 of UGT1 as defined by the position in SEQ ID NO: 1;
the nucleic acid sequence of SEQ ID NO: 2 with G at position 232 in exon 1 of UGT1A6 as defined by the position in SEQ ID NO: 2;
the nucleic acid sequence of SEQ ID NO: 2 with T at position 232 in exon 1 of UGT1A6 as defined by the position in SEQ ID NO: 2;
the nucleic acid sequence of SEQ ID NO: 2 with A at position 528 in exon 1 of UGT1A6 as defined by the position in SEQ ID NO: 2;
the nucleic acid sequence of SEQ ID NO: 2 with G at position 754 in exon 1 of UGT1A6 as defined by the position in SEQ ID NO: 2;
the nucleic acid sequence of SEQ ID NO: 2 with A at position 754 in exon 1 of UGT1A6 as defined by the position in SEQ ID NO: 2;
the nucleic acid sequence of SEQ ID NO: 2 with C at position 765 in exon 1 of UGT1A6 as defined by the position in SEQ ID NO: 2;
the nucleic acid sequence of SEQ ID NO: 2 with A at position 765 in exon 1 of UGT1A6 as defined by the position in SEQ ID NO: 2;
the nucleic acid sequence of SEQ ID NO: 3 with C at position 197 in exon 1 of UGT1A7 as defined by the position in SEQ ID NO: 3;
the nucleic acid sequence of SEQ ID NO: 3 with G at position 551 in exon 1 of UGT1A7 as defined by the position in SEQ ID NO: 3;
the nucleic acid sequence of SEQ ID NO: 3 with T at position 551 in exon 1 of UGT1A7 as defined by the position in SEQ ID NO: 3;
the nucleic acid sequence of SEQ ID NO: 3 with A at position 555 in exon 1 of UGT1A7 as defined by the position in SEQ ID NO: 3;
the nucleic acid sequence of SEQ ID NO: 3 with C at position 555 in exon 1 of UGT1A7 as defined by the position in SEQ ID NO: 3;
the nucleic acid sequence of SEQ ID NO: 3 with A at position 556 in exon 1 of UGT1A7 as defined by the position in SEQ ID NO: 3;
the nucleic acid sequence of SEQ ID NO: 3 with G at position 556 in exon 1 of UGT1A7 as defined by the position in SEQ ID NO: 3;
the nucleic acid sequence of SEQ ID NO: 3 with C at position 786 in exon 1 of UGT1A7 as defined by the position in SEQ ID NO: 3; or
the nucleic acid sequence of SEQ ID NO: 3 with T at position 786 in exon 1 of UGT1A7 as defined by the position in SEQ ID NO: 3;
whenever used in combination with any one of the diagnostic nucleic acids as claimed in claim 7.

9. A set of diagnostic nucleic acids comprising the following polymorphism containing sequences:
the nucleic acid sequence of SEQ ID NO: 1 with C at position 908 in exon 5 as defined by the position in SEQ ID NO: 1;
the nucleic acid sequence of SEQ ID NO: 1 with G at position 908 in exon 5 as defined by the position in SEQ ID NO: 1;
the nucleic acid sequence of SEQ ID NO: 2 with G at position 754 in exon 1 as defined by the position in SEQ ID NO: 2;
the nucleic acid sequence of SEQ ID NO: 2 with A at position 754 in exon 1 as defined by the position in SEQ ID NO: 2;
the nucleic acid sequence of SEQ ID NO: 2 with C at position 765 in exon 1 as defined by
the position in SEQ ID NO: 2;
the nucleic acid sequence of SEQ ID NO: 2 with A at position 765 in exon 1 as defined by the position in SEQ ID NO: 2; or
a complementary strand thereof or a fragment thereof of at least 20 bases comprising at least one of the polymorphisms.

10. A diagnostic nucleic acid primer for detecting a polymorphism in the UGT1 gene capable of hybridizing specifically to a nucleic acid having one of the polymorphisms as defined in claim 4.

11. A diagnostic nucleic acid primer as claimed in claim 10 which is an allele-specific nucleic acid primer having a sequence selected from the group consisting of:
the nucleic acid sequence as defined by SEQ ID NO: 24;
the nucleic acid sequence as defined by SEQ ID NO: 25;
the nucleic acid sequence as defined by SEQ ID NO: 27;
the nucleic acid sequence as defined by SEQ ID NO: 28;
the nucleic acid sequence as defined by SEQ ID NO: 30; or
the nucleic acid sequence as defined by SEQ ID NO: 31.

12. An allele-specific oligonucleotide probe for detecting a polymorphism in the UGT1 gene capable of hybridizing specifically to a nucleic acid having one of the polymorphisms as defined in claim 4.

13. A diagnostic kit comprising one or more diagnostic primer(s) as defined in claim 10 and/or one or more allele-specific oligonucleotide probes(s) as defined in claim 12.

14. A pharmaceutical pack comprising Tolcapone and instructions for administration of the drug to humans diagnostically tested for a single nucleotide polymorphism according to a method as claimed in any one of claims 1 to 5.

15. A computer readable medium having stored thereon sequence information for the polymorphisms in UGT1 at position 908 in exon 5 of the UGT1 gene locus as defined by the position in SEQ ID NO: 1 and/or at position 528 in exon 1 of the UGT1A6 gene as defined by the position in SEQ ID NO: 2 and/or at position 197 in exon 1 of the UGT1A7 gene as defined by the position in SEQ ID NO: 3.

16. A method for performing sequence identification, said method comprising the steps of providing a diagnostic nucleic acid sequence as claimed in any one of claims 6 to 9 and comparing said diagnostic nucleic acid sequence to at least one other nucleic acid or polypeptde sequence to identify identity.

17. A method as hereinbefore described.
